SEQUENCE LISTING (6 sequences)

SEQ ID NO: 1 - protein, 380 aa, Homo sapiens
MVMIISTMEPQVSNGPTSNTSNGPSSNNRNCPSPMQTGATTDDSKTNLIVNYLPQNMTQEEFRSLFGSIGEIESCKLVRDKITGQSLGYGFVNYIDPKDAEKAINTLNGLRLQTKTIKVSYARPSSASIRDANLYVSGLPKTMTQKELEQLFSQYGRIITSRILVDQVTGVSRGVGFIRFDKRIEAEEAIKGLNGQKPSGATEPITVKFANNPSQKSSQALLSQLYQSPNRRYPGPLHHQAQRFRLDNLLNMAYGVKRLMSGPVPPSACPPRFSPITIDGMTSLVGMNIPGHTGTGWCIFVYNLSPDSDESVLWQLFGPFGAVNNVKVIRDFNTNKCKGFGFVTMTNYDEAAMAIASLNGYRLGDRVLQVSFKTNKAHKS

SEQ ID NO: 2 - protein, 6 aa, Artificial Sequence
  Description of Artificial Sequence Synthetic 6xHis tag
HHHHHH

SEQ ID NO: 3 - protein, 15 aa, Artificial Sequence
  Description of Artificial Sequence Synthetic peptide
GGGGSGGGGSGGGGS

SEQ ID NO: 4 - protein, 5 aa, Artificial Sequence
  Description of Artificial Sequence Synthetic peptide
GGGGS

SEQ ID NO: 5 - protein, 14 aa, Artificial Sequence
  Description of Artificial Sequence Synthetic peptide
  MOD_RES (7)..(7): IsoAsp
CTSNTSDGPSSNNR

SEQ ID NO: 6 - protein, 117 aa, Homo sapiens
MEPQVSNGPTSNTSNGPSSNNRNCPSPMQTGATTDDSKTNLIVNYLPQNMTQEEFRSLFGSIGEIESCKLVRDKITGQSLGYGFVNYIDPKDAEKAINTLNGLRLQTKTIKVSYARP

(19) United States
(12) Patent Application Publication
(10) Pub. No.: US 2019/0290758 A1
(43) Pub. Date: Sep. 26, 2019

(54) COMPOSITIONS AND METHODS FOR THE TREATMENT OF CANCER

(71) Applicants: Ite Offringa, Los Angeles, CA (US); University of Southern California, Los Angeles, CA (US)

(72) Inventor: Ite Offringa, Los Angeles, CA (US)

(21) Appl. No.: 16/345,210

(22) PCT Filed: Oct. 30, 2017
(86) PCT No.: PCT/US2017/059007

§ 371 Date: Apr. 25, 2019

Related U.S. Application Data
(60) Provisional application No. 62/414,587, filed on Oct. 28, 2016.

(51) Int. Cl. A61K 39/395; G01N 33/574; C07K 14/47; C07K 16/30; A61P 35/00

(52) U.S. Cl. CPC A61K 39/39558; G01N 33/57484; A61P 35/00; C07K 16/30; C07K 14/4748

(57) ABSTRACT

This disclosure provides compositions and methods for treating cancer or inducing an immune response to the cancer in a subject by administering to the subject an effective amount of an isoaspartylated protein or a fragment thereof or an antibody that binds specifically to an isoaspartylated protein or a fragment thereof. In another aspect, the disclosure also provides a pharmaceutical composition, comprising, consisting essentially of, or yet further consisting of an effective amount of the isoaspartylated protein or a fragment thereof or antibody that binds specifically to an isoaspartylated protein or a fragment thereof; and a pharmaceutically acceptable carrier.

### CROSS REFERENCE TO RELATED APPLICATION

This application claims priority under 35 U.S.C. § 119(e) to U.S. Provisional Application No. 62/414,587, filed Oct. 28, 2016, the content of which is hereby incorporated by reference in its entirety.

### STATEMENT OF GOVERNMENT SUPPORT

This invention was supported in part by a Grant D2016-0163 awarded by the US Department of Defense. Accordingly, the US government has rights in this invention.

### BACKGROUND

Small cell lung cancer (SCLC) is the most aggressive lung cancer subtype, with an overall 5-year survival of only 6% (Howlader et al., 2013). Aside from prophylactic cranial irradiation to limit brain metastases, there have been no meaningful therapeutic advances in three decades (Kalemkerian, 2014). The existence of multiple SCLC-associated autoimmune syndromes suggests an interplay between SCLC and the immune system. SCLC patients with autoimmune syndromes exhibit high titer antibodies against neuronal proteins ectopically expressed by their tumors and can show regression (Dalmau et al., 1992; Winter et al., 1993; Darnell and DeAngelis, 1993; Graus et al., 1997; Honnorat et al., 2009; Kobayashi et al., 2007; Kazarian and Laird-Offringa, 2011). Although the high titer patients are rare, the characteristic autoantibodies are found in a substantial fraction of SCLC patients without autoimmune symptoms, albeit at lower titers (Graus et al., 1997; Kazarian and Laird-Offringa, 2011; Dalmau et al., 1990). Thus, while an immune response is relatively common, it does not usually progress to a paraneoplastic disease. Thus, a need exists in the art for safe and effective therapies against SCLC and metastases. This disclosure satisfies that need and provides related advantages as well

### SUMMARY OF THE DISCLOSURE

Autoantibodies in SCLC patients have been associated with improved survival, suggesting that the immune response could be harnessed therapeutically. Understanding the mechanism triggering SCLC-associated immune responses may provide new tools for SCLC detection, diagnosis and new treatments, such as immunotherapy (Kazarian and Laird-Offringa, 2011).

One well-known family of proteins that can become self antigens in SCLC is that of the neuronal embryonic lethal altered visual system-like (ELAVL) RNA-binding proteins (formerly “Hu” proteins), which are expressed in every SCLC, but not in non-SCLC (Manley et al., 1995). Antibodies to neuronal ELAVL correlate with improved survival of SCLC patients (Graus et al., 1997). Furthermore, in patients with full-blown anti-ELAVL autoimmune disease (paraneoplastic encephalomyelitis/sensory neuropathy, or PEM/SN), the tumors are often small and localized (Dalmau et al., 1992). Of the neuronal ELAVL proteins, tumors most commonly express ELAVL4 (HuD) (Manley et al., 1995). The highly homologous ELAVL2 (HuB/Hel-N1) and ELAVL3 (HuC) are also neuronal, while the less conserved ELAVL1 (HuR) is ubiquitously expressed. Although less than 1% of SCLC patients develop high titer anti-ELAVL antibodies and exhibit PEM/SN, lower titer antibodies are seen in about 15-20% of SCLC patients without autoimmune symptoms (Graus et al., 1997; Kazarian and Laird-Offringa, 2011; Dalmau et al., 1990). How this immune response develops remains in question. There is little evidence for immunogenic mutations (Sekido et al., 1994; Carpentier et al., 1998; D'Alessandro et al., 2010). Without being bound by theory, Applicant hypothesized that fased on the sequence and presumably unstructured nature of the N-terminal region of neuronal ELAVL proteins, that in the context of SCLC these proteins can undergo isoaspartylation, a naturally occurring immunogenic post-translational modification. Isoaspartyl moieties are normally repaired in the body, and abnormal isoaspartylation been implicated in several autoimmune diseases (Mamula et al., 1999; Yang et al., 2006).

Thus, building on these discoveries, provided herein is an isolated an isoaspartylated protein or a fragment thereof, (e.g., an antigenic Hu polypeptide), the protein consisting of a protein fragment and its use in and as as a vaccine or therapeutic composition and in generating antibodies, e.g., polyclonal and monoclonal antibodies, for diagnostic and therapeutic use. In one embodiment, the protein is is an embryonic lethal altered visual system-like RNA-binding protein (Hu protein, e.g., HuD protein). In another embodiment, the protein comprises, consists essentially of, or yet further consists of the HuD protein or fragment thereof (e.g., a N-terminal fragment comprising ELAVL4₁₋₁₁₇ or an equivalent of each thereof) comprising an isoaspartylated residue. In some embodiment, the fragment comprises, consists essentially of, or yet further consists of an amino acid capable of isoAsp conversion. In a further embodiment, the fragment comprises, consists essentially of, or yet further consists of an isoaspartylated residue. In one embodiment, the fragment comprises, consists essentially of, or yet further consists of an RNA recognition motif 1 (RRM1) or an equivalent thereof. The polypeptides, proteins and fragments can be further combined with a carrier, such as a pharmaceutically acceptable carrier and an adjuvant.

Also provided are polynucleotides that encode the an isoaspartylated protein or a fragment thereof, (e.g., an antigenic Hu polypeptide) as described herein. The polynucleotides and/or polypeptides are optionally labeled with a detectable or purification label, wherein in one aspect, the detectable label is not a continguous naturally occurring nucleic acid. Also provided are host cells containing these polynucleotides and/or polypeptides and use of the cells for recombinant production of the compositions. Further provided are host vector systems comprising the polynucleotides operatively linked with the appropriate regulatory elements, e.g., promoter and/or enhancer into a replication vector or another insertion vector. These compositions can be combined with a carrier or an adjuvant, such as a pharmaceutically acceptable carrier.

The disclosure also provides a pharmaceutical composition comprising, consisting essentially of, or yet further consisting, of an effective amount of an isoaspartylated protein or a fragment thereof, or an antibody or antigen binding fragment that binds specifically to an isoaspartylated protein or a fragment thereof vectors and host cells as described herein, and a pharmaceutically acceptable carrier and/or adjuvant. In one embodiment, the protein is a HuD protein and the antigenic fragment is a fragment of HuD. In some embodiment, the protein or fragment thereof comprises, consists essentially of, or yet further consists of an isoaspartylated residue. In a different embodiment, the protein or fragment comprises, consists essentially of, or yet further consists of an amino acid capable of isoAsp conversion. In a further embodiment, the protein or fragment comprises, consists essentially of, or yet further consists of an isoaspartylated residue. In some embodiment, the protein or fragment comprises, consists essentially of, or yet further consists of an RNA recognition motif 1 (RRM1).

In some embodiment, the antibody is a polyclonal or a monoclonal antibody. In a related embodiment, the monoclonal antibody is humanized or specisized. In another aspect, an antigen binding fragment of the antibody is utilized. In one aspect, the antibodies or antigen binding fragments are labeled with a detectable or a purification label.

Also provided are polynucleotides encoding the antibodies and antigen binding fragments that are optionally labeled with a detectable or purification label, wherein in one aspect, the detectable label is not a continguous naturally occurring nucleic acid. Also provided are host cells containing these polynucleotides and/or polypeptides and use of the cells for recombinant production of these compositions. Further provided are host vector systems comprising the polynucleotides, e.g., a replication or another insertion vector. These compositions can be combined with a carrier, such as a pharmaceutically acceptable carrier and/or an adjuvant. Further provided are methods for generating these antibodies and antigen binding fragments.

This disclosure provides a method of treating cancer or inducing an immune response to the cancer, e.g. a solid tumor, non-limiting examples of such that include cancers in tissues that would not normally express neuronal proteins, e.g., small cell lung cancer (SCLC), breast cancer, melanoma, and gynecological cancers in a subject, comprising, consisting essentially of, or yet further consisting of administering to the subject an effective amount of an isolated isoaspartylated protein or a fragment thereof, (e.g., an antigenic Hu polypeptide). Also provide is the protein consisting of a protein fragment and its use as a vaccine or therapeutic composition and in generating antibodies, e.g., polyclonal and monoclonal antibodies, for diagnostic and therapeutic use. In one embodiment, the protein is is an embryonic lethal altered visual system-like RNA-binding protein (Hu protein, e.g., HuD protein). In another embodiment, the protein comprises, consists essentially of, or yet further consists of the HuD protein or fragment thereof (e.g., a N-terminal fragment comprising ELAVL4₁₋₁₁₇ or an equivalent thereof) an isoaspartylated residue. In some embodiment, the fragment comprises, consists essentially of, or yet further consists of an amino acid capable of isoAsp conversion. In a further embodiment, the fragment comprises, consists essentially of, or yet further consists of an isoaspartylated residue. In one embodiment, the fragment comprises, consists essentially of, or yet further consists of an RNA recognition motif 1 (RRM1) or an equivalent thereof. The polypeptides, proteins and fragments can be further combined with a carrier, such as a pharmaceutically acceptable carrier. An effective amount of the compositions are administered in once, twice, or thrice or more, and as first-line, second-line, third-line, fourth-line, or other subsequent follow-on therapies. The subject can be an animal and therefore used as an animal model to test therapies that could be used in combination with the compositions as disclosed herein. In another aspect, the subject is a human patient in need thereof, having been diagnosed with a cancer as described above. Methods to determine if the treatment is successful include clinical responses such as a reduction in tumor size or burden, or the eliciting antibodies or cellular immunity.

This disclosure provides a method of treating cancer, e.g. a solid tumor, non-limiting examples of such that include cancers in tissues that would not normally express neuronal proteins, e.g., small cell lung cancer (SCLC), breast cancer, melanoma, and gynecological cancers in a subject, comprising, consisting essentially of, or yet further consisting of administering to the subject an effective amount of an antibody that binds specifically to an isoaspartylated protein or a fragment thereof. In one embodiment, the protein is is an embryonic lethal altered visual system-like RNA-binding protein (Hu protein, e.g., HuD protein). In another embodiment, the protein comprises, consists essentially of, or yet further consists of the HuD protein or fragment thereof (e.g., a N-terminal fragment comprising ELAVL4₁₋₁₁₇ or an equivalent thereof) an isoaspartylated residue. In some embodiment, the fragment comprises, consists essentially of, or yet further consists of an amino acid capable of isoAsp conversion. In a further embodiment, the fragment comprises, consists essentially of, or yet further consists of an isoaspartylated residue. In one embodiment, the fragment comprises, consists essentially of, or yet further consists of an RNA recognition motif 1 (RRM1) or an equivalent thereof. An effective amount of the compositions are administered in once, twice, or thrice or more, and as first-line, second-line, third-line, fourth-line, or other subsequent follow-on therapies. The subject can be an animal and therefore used as an animal model to test therapies that could be used in combination with the compositions as disclosed herein. In another aspect, the subject is a human patient in need thereof, having been diagnosed with a cancer as described above. Methods to determine if the treatment is successful include clinical responses such as a reduction in tumor size or burden, or the eliciting antibodies or cellular immunity.

In some embodiment, the antibody is a monoclonal antibody or an antigen binding fragment of the antibody is used. In some embodiment, the monoclonal antibody is humanized or the antigen binding fragment is derived from a humanized antibody. In a different embodiment, the antibody is a polyclonal antibody.

### BRIEF DESCRIPTION OF THE FIGURES

The drawings as presented herein set forth various aspects of the invention as further described herein.

FIGS. 1A-1C show that ELAVL4 N-terminal region becomes isoaspartylated in vitro. FIG. 1A shows the mechanism of isoAsp formation and repair. Isoaspartylation occurs by spontaneous dehydration or deamidation of an asparagine (Asn-Xaa) or aspartate (Asp-Xaa) linkage (where Xaa is usually glycine, serine or histidine). This produces a metastable succinimide intermediate, which in turn undergoes spontaneous hydrolysis to generate an unequal mixture, typically 15-30% of the normal L-aspartyl linkage and 70-85% of an abnormal L-isoaspartyl-Xaa linkage with a kinked protein backbone (protein backbone indicated in black). PIMT converts L-isoaspartyl sites to α-carboxyl-O-methyl esters, converting methyl donor S-adenosyl methionine (SAM) to S-adenosyl homocysteine (SAH). At physiological pH and temperature, the α-carboxyl-O-methyl esters have a typical half-life of a few minutes and undergo spontaneous demethylation to reform the succinimide intermediate. Each PIMT cycle typically repairs ˜25% of the isoAsp peptide bond by converting it to a normal aspartyl peptide. Unrepaired peptides are continuously recycled, and the overall repair efficiency is 85% or greater. In FIG. 1B, canonical isoAsp sites (N or D followed by S, G or H in unstructured regions) are boxed, all N and D with potential for isoaspartylation are bolded. Without being bound by theory, Applicants opine that it is unlikely that the globular structure of the RRM1 domain (dashed box) allows isoaspartylation of RRM1 residues. The isoAsp-containing peptide used to immunize rabbits is underlined and homologous amino acids shared between this isoAsp-containing peptide and the peptide sequence surrounding N7 are starred. In the top panel of FIG. 1C, the recombinant wild type ELAVL4₁₋₁₁₇ protein, a mutant with substitutions at all N (to Q) and D (to E) residues in the N-terminal 38 amino acids (indicated by the dashed line), and ELAVL4₃₉₋₁₁₇, a protein without the N-terminal region, were incubated at physiological conditions for 0, 3, and 7 days, separated by gel electrophoresis, transferred to a membrane and subjected to on-blot labeling with PIMT and ³H-SAM (top panel). Only wild type ELAVL4₁₋₁₁₇ showed above background labeling by PIMT at 3 and 7 days. The bottom panel of FIG. 1C shows a Coomassie-stained gel, indicating similar loading of wild type, mutant, and truncated proteins over the incubation period. A representative image of several similar experiments is shown.

FIGS. 2A-2D shows that an anti-isoAsp ELAVL4 antiserum detects isoaspartylation in vitro. FIG. 2A shows a schematic representation of polyclonal rabbit anti-isoAsp ELAVL4 antiserum purification. The labels a-d match those in the histogram in panel B. FIG. 2B is an ELISA analysis of pre-purified serum (a, 1:100,000 dilution), affinity purification flow-through (b, 1:100,000 dilution), affinity purified eluted antibody (c, 0.01 μg/m1), affinity-absorbed antibody (d, 0.01 μg/m1), and no antibody controls (leftmost white and grey bars) against an unmodified ELAVL4 peptide and the same peptide containing an isoaspartate residue at N₁₅ (see FIG. 1B, underlined). ELISA data provided by YenZym Antibodies, South San Francisco, Calif. In FIG. 2C, the isoAsp-ELAVL4 antisera specifically recognizes in vitro isoAsp-converted ELAVL4₁₋₁₁₇. 2 μg wild type, single mutant N₁₅Q and double mutant N₇Q+N₁₅Q protein per lane were in vitro isoaspartyl converted at 37° C. for up to seven days. Samples from day 0, 1, 3, and 7 were subjected to SDS-PAGE and Western blot analysis and exposed for 5 minutes. A representative example of triplicate experiments is shown. (See Pulido et al. 2016 for reactivity of antiserum with other ELAVL family members.) FIG. 2D shows the results of a competition assay using the same ELAVL4 peptide against which the rabbit antisera had been raised, in its native or isoaspartylated form.

FIGS. 3A-3C shows that small cell lung cancer antigen ELAV is prone to isoaspartylation in vivo. In FIG. 3A, brain extract from PIMT wild type and knock out mice was subjected to Western blot analysis. The brain is the normal site of ELAVL4 expression. Blots were probed with anti-PIMT and anti-ELAVL4 antibodies and the rabbit affinity-purified anti-isoAsp ELAVL4 antiserum. Membranes were stripped and reprobed using an anti-actin antibody to check for similar loading. Exposure time was 45 seconds. FIG. 3B shows quantitation of isoAsp-ELAVL4 reactivity. FIG. 3C (top and bottom panels) show anti-ELAVL4 reactivity of human archival SCLC tumors. Two representative cases are shown out of 5 examined. De-identified archival paraffin blocks were sectioned and subjected to H&E staining or immunohistochemistry with antisera to ELAVL4, isoAsp-ELAVL4, and PIMT. Specificity of the anti-ELAVL4 antiserum was assessed by competition with excess native or isoAsp-converted ELAVL4₁₋₁₁₇. Asterisks and circles are provided for sample orientation purposes. Top panel, magnification of sections 10×; lower panel, 20×.

FIGS. 4A-4E show that isoaspartylated ELAVL4₁₋₁₁₇ is highly immunogenic. FIG. 4A schematic depicts a strategy to assess the immunogenic potential of isoaspartylated-ELAVL4 in mice. Three mice were each immunized with native ELAVL4₁₋₁₁₇, the same protein incubated under isoaspartylation conditions for 7 days, or PBS were used. In FIG. 4B, anti-ELAVL4 and anti-isoAsp-ELAVL4 reactivity of mouse plasma was determined as in FIG. 2C by western blot analysis using 0.25 μg recombinant protein in its native form (Nat) or incubated under isoapsartylation conditions for 7 days (Iso); dilutions of mouse plasma are indicated at top (post-boosting sera were more diluted). The right-most panel shows rehybridization of the strips from mouse 525 with an anti-(His)₆ tag antibody. FIG. 4C is the quantification of mouse antibody response. FIG. 4D shows T-cell proliferation comparing mice immunized with native vs. isoAsp-ELAVL4₁₋₁₁₇. FIG. 4E shows induction of human immune cell proliferation in vitro with isoaspartylated ELAVL4₁₋₁₁₇. Human PBMC from four unidentified donors were incubated in triplicate with native ELAVL4₁₋₁₁₇, isoAsp-ELAVL4₁₋₁₁₇ or positive control phytohemagglutinin (PHA) 72 h prior to [³H]thymidine pulse. Total cell proliferation was then measured at 96 h. This graph is a representative example of an experiment. * p<0.05, *** p<0.001.

FIG. 5 shows that SCLC patient sera target the isoAsp-prone region. Recombinant ELAVL4₁₋₁₁₇ and RRM1 alone were subjected to isoAsp-inducing conditions and gel electrophoresis. The top left panel shows a Coomassie stain of a representative gel. As the protein acquires isoaspartylation (Day 3, 7), mobility changes, widening the band on gel. The other panels show blots probed with patient antiserum (top section) or stripped and reprobed with an anti-(His)₆ antibody (1:10,000-20,000) recognizing the C-terminal hexahistidine tag on the recombinant proteins (only bottom section of each panel is shown to indicate protein presence). Top row: 1:10,000 human serum dilution. Bottom row 1:1,000 human serum dilution, except for patient 7, 1:500 dilution. Note the weak or absent staining of the RRM1 alone fragment with the human sera.

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds LHR will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen binding domain” refers to any protein or polypeptide domain that can specifically bind to an antigen target.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further non-limiting examples of such domains are provided herein.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. It is to be understood that the vectors contain the necessary regulatory elements for replication or expression of the inserted polynucleotide, including for example promoters or enhancer elements.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “isolated cell” generally refers to a cell that is substantially separated from other cells of a tissue. “Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses.

The term “transduce” or “transduction” as it is applied to the production of chimeric antigen receptor cells refers to the process whereby a foreign nucleotide sequence is introduced into a cell. In some embodiments, this transduction is done via a vector.

As used herein, the term “autologous,” in reference to cells refers to cells that are isolated and infused back into the same subject (recipient or host). “Allogeneic” refers to non-autologous cells.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

A host cell can be a eukaryotic or a prokaryotic cell. “Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

As used herein, the term “purification label” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, infusion, injection, and topical application. As understood by those of skill in the art, the therapies can be co-administered with other therapies, such as immuno-oncology or chemotherapy. The therapies can be administered simultaneously or concurrently.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸ M, 10⁻⁹ M, 10⁻¹⁰ M, 10⁻¹¹, or 10⁻¹² M.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

As used herein the term “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 flexible polypeptide linker comprising three copies of gly-gly-gly-gly-ser.

As used herein, the term “enhancer”, as used herein, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

### Descriptive Embodiments

Autoantibodies against SCLC-associated neuronal antigen ELAVL4 (HuD) (an exemplary disclosure of HuD is found at UniProtKK—P26378 and SEQ ID NO.: 1, available at the web address: uniprot.org/uniprot/P26378, last accessed on Oct. 28, 2016) have been linked to smaller tumors and improved survival, but the antigenic epitope and mechanism of autoimmunity have never been solved. As reported herein, recombinant human ELAVL4 protein incubated under physiological conditions acquires isoaspartylation, a type of immunogenic protein damage. Specifically, the N-terminal region of ELAVL4, previously implicated in SCLC-associated autoimmunity, undergoes isoaspartylation in vitro, is recognized by sera from anti-ELAVL4 positive SCLC patients and is highly immunogenic in subcutaneously injected mice and in vitro stimulated human lymphocytes. The data reported herein suggests that isoaspartylated ELAVL4 is the trigger for the SCLC-associated anti-ELAVL4 autoimmune response.

### Protein and Polypeptide Compositions

In one aspect, provided herein is an isolated isoaspartylated protein or a fragment thereof, (e.g., an antigenic Hu polypeptide). In one aspect, the protein consists essentially of or consists of a protein fragment and its use as a vaccine or therapeutic composition and in eliciting an anticancer immune response and for generating antibodies, e.g., polyclonal and monoclonal antibodies, for diagnostic and therapeutic use. In one embodiment, the protein is is an embryonic lethal altered visual system-like RNA-binding protein (Hu protein, e.g., HuD protein). In another embodiment, the protein comprises, consists essentially of, or yet further consists of the HuD protein or fragment thereof (e.g., a N-terminal fragment comprising ELAVL4₁₋₁₁₇ or an equivalent thereof) an isoaspartylated residue. In some embodiment, the fragment comprises, consists essentially of, or yet further consists of an amino acid capable of isoAsp conversion. In a further embodiment, the fragment comprises, consists essentially of, or yet further consists of an isoaspartylated residue. In one embodiment, the fragment comprises, consists essentially of, or yet further consists of an RNA recognition motif 1 (RRM1) or an equivalent thereof. The polypeptides, proteins and fragments can be further combined with a carrier, such as a pharmaceutically acceptable carrier. In a further aspect, the polypetides, proteins and fragments thereof are combined with a detectable or a purification label.

Further provided is a fragment or an equivalent of the isolated or recombinant polypeptide of any one of polypeptides identified above as well as an isolated or recombinant polypeptide comprising, or alternatively consisting essentially of, or yet further consisting of, two or more of the isolated or recombinant polypeptides identified above.

Also provided is an isolated host cell comprising, or alternatively consisting essentially of, or yet further consisting of an isolated or recombinant polypeptide described above. The host cell is prokaryotic or eukaryotic.

The proteins and polypeptides can be combined with a carrier, such as a pharmaceutical acceptable carrier. In a further aspect, they are combined with stabalizers and/or preservatives that allow stable formulations. In one aspect the compositions are lyophilized or freeze-dried for ease of transport ad storage. They also can comprise an adjuvant when used as a vaccine composition.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

Also provided are pharmaceutical formulations comprising an effective amount of the protein or polypeptide. The formulations can be provided in kits comprising the formulations and instructions for use. The formulations can be lyophilized for ease of storage, transport and use.

Also provided are the polynucleotides encoding the polypeptides, recombinant expression systems and host cells comprising the polynucleotides and use of same for the recombinant expression of the polypeptides as well as the recombinant polypeptides encoding by the system and host cells. In one aspect, the host cell is a mammalian cell. The isolated polynucleotides can be operatively linked to regulatory elements necessary for the expression and/or replication of the polynucleotide. The polynucleotide can be contained within a vector. In one aspect, the polynucleotides further comprise an artificial or non-naturally occurring label (e.g., excluding naturally fluorescent polynucleotides) bound to the polynucleotide for use as probes for use in detection of antibodies.

The disclosure further provides the isolated or recombinant polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are known in the art and commercially available. For general methodology and cloning strategies, see Gene Expression Technology (Goeddel ed., Academic Press, Inc. (1991)) and references cited therein and Vectors: Essential Data Series (Gacesa and Ramji, eds., John Wiley & Sons, N.Y. (1994)) which contains maps, functional properties, commercial suppliers and a reference to GenEMBL accession numbers for various suitable vectors.

In one embodiment, polynucleotides derived from the polynucleotides disclosed herein encode polypeptides or proteins having diagnostic and therapeutic utilities as described herein as well as probes to identify transcripts of the protein that may or may not be present. These nucleic acid fragments can by prepared, for example, by restriction enzyme digestion of larger polynucleotides and then labeled with a detectable marker. Alternatively, random fragments can be generated using nick translation of the molecule. For methodology for the preparation and labeling of such fragments, see, Sambrook et al. (1989) supra.

This disclosure also provides genetically modified cells that contain and/or express the polynucleotides disclosed herein. The genetically modified cells can be produced by insertion of upstream regulatory sequences such as promoters or gene activators (see, U.S. Pat. No. 5,733,761).

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In one aspect, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates can be employed to provide a means visible to the human eye or spectrophotometrically, to identify specific hybridization with complementary nucleic acid-containing samples. Thus, this disclosure further provides a method for detecting a single-stranded polynucleotide or its complement, by contacting target single-stranded polynucleotide with a labeled, single-stranded polynucleotide (a probe) which is a portion of the polynucleotide disclosed herein under conditions permitting hybridization (optionally moderately stringent hybridization conditions) of complementary single-stranded polynucleotides, or optionally, under highly stringent hybridization conditions. Hybridized polynucleotide pairs are separated from un-hybridized, single-stranded polynucleotides. The hybridized polynucleotide pairs are detected using methods known to those of skill in the art and set forth, for example, in Sambrook et al. (1989) supra.

Expression vectors containing these nucleic acids are useful to obtain host vector systems to produce proteins and polypeptides. It is implied that these expression vectors must be replicable in the host organisms either as episomes or as an integral part of the chromosomal DNA. Non-limiting examples of suitable expression vectors include plasmids, yeast vectors, viral vectors and liposomes. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using known methods. See, Sambrook et al. (1989) supra. In addition to the use of viral vector for insertion of exogenous nucleic acid into cells, the nucleic acid can be inserted into the host cell by methods known in the art such as transformation for bacterial cells; transfection using calcium phosphate precipitation for mammalian cells; or DEAE-dextran; electroporation; or microinjection. See, Sambrook et al. (1989) supra, for methodology. Thus, this disclosure also provides a host cell, e.g., a mammalian cell, an animal cell (rat or mouse), a human cell, or a prokaryotic cell such as a bacterial cell, containing a polynucleotide encoding a protein or polypeptide or antibody.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

This disclosure also provides isolated or recombinant polynucleotides encoding one or more of the above-identified isolated or recombinant polypeptides and their respective complementary strands. Vectors comprising the isolated or recombinant polynucleotides are further provided examples of which are known in the art and briefly described herein.

A polynucleotide as disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a polynucleotide.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

The proteins and polypeptides are obtainable by a number of processes known to those of skill in the art, which include purification, chemical synthesis and recombinant methods. Polypeptides can be isolated from preparations such as host cell systems by methods such as immunoprecipitation with antibody, and standard techniques such as gel filtration, ion-exchange, reversed-phase, and affinity chromatography. For such methodology, see for example Deutscher et al. (1999) Guide To Protein Purification: Methods In Enzymology (Vol. 182, Academic Press). Accordingly, this disclosure also provides the processes for obtaining these polypeptides as well as the products obtainable and obtained by these processes.

The polypeptides also can be obtained by chemical synthesis using a commercially available automated peptide synthesizer such as those manufactured by Perkin/Elmer/Applied Biosystems, Inc., Model 430A or 431A, Foster City, Calif., USA. The synthesized polypeptide can be precipitated and further purified, for example by high performance liquid chromatography (HPLC). Accordingly, this disclosure also provides a process for chemically synthesizing the proteins disclosed herein by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al. (1989) supra, using a host cell and vector systems described herein.

Also provided by this application are the polypeptides described herein conjugated to a detectable agent for use in the diagnostic methods. For example, detectably labeled polypeptides can be bound to a column and used for the detection and purification of antibodies. They also are useful as immunogens for the production of antibodies as described below. The polypeptides disclosed herein are useful in an in vitro assay system to screen for agents or drugs, which modulate cellular processes.

It is well known to those skilled in the art that modifications can be made to the peptides disclosed herein to provide them with altered properties. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

Peptides disclosed herein can be modified to include unnatural amino acids. Thus, the peptides may comprise D-amino acids, a combination of and L-amino acids, and various “designer” amino acids (e.g., .beta.-methyl amino acids, C-alpha-methyl amino acids, and N-alpha-methyl amino acids, etc.) to convey special properties to peptides. Additionally, by assigning specific amino acids at specific coupling steps, peptides with alpha-helices, beta. turns, beta. sheets, gamma-turns, and cyclic peptides can be generated. Generally, it is believed that .alpha.-helical secondary structure or random secondary structure may be of particular use.

The polypeptides disclosed herein also can be combined with various solid phase carriers, such as an implant, a stent, a paste, a gel, or a medical implant or liquid phase carriers, such as beads, sterile or aqueous solutions, pharmaceutically acceptable carriers, pharmaceutically acceptable polymers, liposomes, micelles, suspensions and emulsions. Examples of non-aqueous solvents include propyl ethylene glycol, polyethylene glycol and vegetable oils. When used to prepare antibodies or induce an immune response in vivo, the carriers also can include an adjuvant that is useful to non-specifically augment a specific immune response. A skilled artisan can easily determine whether an adjuvant is required and select one. However, for the purpose of illustration only, suitable adjuvants include, but are not limited to Freund's Complete and Incomplete, mineral salts and polynucleotides. Other suitable adjuvants include monophosphoryl lipid A (MPL), mutant derivatives of the heat labile enterotoxin of E. coli, mutant derivatives of cholera toxin, CPG oligonucleotides, and adjuvants derived from squalene.

### Methods of Treatment

This disclosure also provides a method of treating a cancer or inducing an immune response to the cancer in a subject in need thereof, the method comprising, or alternatively consisting essentially of, or yet further consisting of, administering to the subject an effective amount of a therapeutic composition as described herein, e.g., an isoaspartylated protein or a fragment thereof as described herein, thereby treating the cancer or inducing an immune response to the cancer. In one aspect, the the cancer is a solid tumor. In another aspect, the cancer is a cancer in tissues that would not normally express neuronal proteins. Non-limiting examples of such include a cancer selected from small cell lung cancer (SCLC), breast cancer, melanoma, and a gynecological cancer.

The subject is an animal such as a sport animal, a pet or a farm animal. In another aspect, the subject is a human patient. One of skill in the art can determine when the treatment has been effective by assaying a sample isolated from the subject for antibodies that specifically recognize and bind the protein or fragment thereof, or by noting a cellular response.

In one aspect, the protein is an embryonic lethal altered visual system-like RNA-binding protein (Hu protein, optionally HuD protein). In another aspect, the protein comprises an isoaspartylated residue. In a further aspect, the fragment comprises an amino acid capable of isoAsp conversion. In a yet further aspect, the fragment comprises one or more isoaspartylated residue. In a yet further aspect, the fragment comprises an RNA recognition motif 1 (RRM1).

The therapy can be combined with other anti-cancer therapies which can be administered simultaneously or in sequence as determined by the treating physician or veterinarian.

### Antibodies

The general structure of antibodies is known in the art and will only be briefly summarized here. An immunoglobulin monomer comprises two heavy chains and two light chains connected by disulfide bonds. Each heavy chain is paired with one of the light chains to which it is directly bound via a disulfide bond. Each heavy chain comprises a constant region (which varies depending on the isotype of the antibody) and a variable region. The variable region comprises three hypervariable regions (or complementarity determining regions) which are designated CDRH1, CDRH2 and CDRH3 and which are supported within framework regions. Each light chain comprises a constant region and a variable region, with the variable region comprising three hypervariable regions (designated CDRL1, CDRL2 and CDRL3) supported by framework regions in an analogous manner to the variable region of the heavy chain.

The hypervariable regions of each pair of heavy and light chains mutually cooperate to provide an antigen binding site that is capable of binding a target antigen. The binding specificity of a pair of heavy and light chains is defined by the sequence of CDR1, CDR2 and CDR3 of the heavy and light chains. Thus once a set of CDR sequences (i.e. the sequence of CDR1, CDR2 and CDR3 for the heavy and light chains) is determined which gives rise to a particular binding specificity, the set of CDR sequences can, in principle, be inserted into the appropriate positions within any other antibody framework regions linked with any antibody constant regions in order to provide a different antibody with the same antigen binding specificity.

In one aspect, the present disclosure provides an isolated antibody comprising a heavy chain (HC) immunoglobulin variable domain sequence and a light chain (LC) immunoglobulin variable domain sequence, wherein the heavy chain and light chain immunoglobulin variable domain sequences form an antigen binding site that binds to the antigenic fragment, e.g., an antigenic fragment of HuD, e.g., ELAVL4₁₋₁₁₇ or an equivalent thereof.

In some of the aspects of the antibodies provided herein, the antibody binds the fragment with a dissociation constant (K_{D}) of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M.

In some of the aspects of the antibodies provided herein, the antibody is antigen binding fragment such as a soluble Fab.

In some of the aspects of the antibodies provided herein, the antibody is a full-length antibody.

In some of the aspects of the antibodies provided herein, the antibody is a monoclonal antibody.

In some of the aspects of the antibodies provided herein, the antibody is chimeric, humanized or speciesized as well as antigen binding fragments thereof.

In some of the aspects of the antibodies provided herein, the antibody is selected from the group consisting of Fab, F(ab)′2, Fab′, scFᵥ, and Fᵥ.

In some of the aspects of the antibodies provided herein, the antibody comprises an Fc domain. In some of the aspects of the antibodies provided herein, the antibody is a rabbit antibody. In some of the aspects of the antibodies provided herein, the antibody is a human or humanized antibody or is non-immunogenic in a human.

In some of the aspects of the antibodies provided herein, the antibody comprises a human antibody framework region.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families.
1) Amino acids with basic side chains: lysine, arginine, histidine.
2) Amino acids with acidic side chains: aspartic acid, glutamic acid
3) Amino acids with uncharged polar side chains: asparagine, glutamine, serine, threonine, tyrosine.
4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N-or C-termini of the CDR or at a position within the CDR.

By varying the amino acid sequence of the CDRs of an antibody by addition, deletion or substitution of amino acids, various effects such as increased binding affinity for the target antigen may be obtained.

It is to be appreciated that antibodies of the present disclosure comprising such varied CDR sequences still bind the antigenic fragment with similar specificity and sensitivity profiles as the disclosed antibodies. This may be tested by way of the binding assays.

The constant regions of antibodies may also be varied. For example, antibodies may be provided with Fc regions of any isotype: IgA (IgA1, IgA2), IgD, IgE, IgG (IgG1, IgG2, IgG3, IgG4) or IgM. Non-limiting examples of constant region sequences include:

In some aspects of the antibodies provided herein, the antibody contains structural modifications to facilitate rapid binding and cell uptake and/or slow release. In some aspects, the antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

The antibodies, fragments, and equivalents thereof can be combined with a carrier, e.g., a pharmaceutically acceptable carrier or other agents to provide a formulation for use and/or storage.

Further provided are antibodies and antigen binding fragments that bind to an isolated polypeptide comprising, or alternatively consisting essentially of, or yet further consisting of, the amino acid sequence of an antigenic fragment of HuD, e.g., ELAVL4₁₋₁₁₇ or an equivalent thereof. In one aspect the antibodies and antigen binding fragments thereof a bind to an antigenic fragment of HuD, e.g., ELAVL4₁₋₁₁₇ or an equivalent thereof. In one aspect, the antibodies and antigen binding fragments further comprise a label and/or contiguous polypeptide sequences (e.g., keyhole limpet haemocyanin (KLH) carrier protein). The antibodies and fragment thereof can be combined with various carriers, e.g., phosphate buffered saline. Further provided are polynucleotides encoding the antibodies or antigen binding fragments alone or contained within host cells, e.g., prokaryotic or eukaryotic cells, e.g., bacteria, yeast, mammalian (rat, simian, hamster, or human). The host cells can be combined with a carrier.

### Processes for Preparing Antibodies and Antibody Compositions

Antibodies, their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties, such as a C-terminal fragment of antigenic HuD or an isolated polypeptide. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This this disclosure also provides the isolated polypeptide and an adjuvant.

In certain aspects, the antibodies of the present disclosure are polyclonal, i.e., a mixture of plural types of anti HuD, e.g., anti-ELAVL4₁₋₁₁₇ antibodies having different amino acid sequences. In one aspect, the polyclonal antibody comprises a mixture of plural types of anti HuD, e.g., anti-ELAVL4₁₋₁₁₇ antibodies having different CDRs. As such, a mixture of cells which produce different antibodies is cultured, and an antibody purified from the resulting culture can be used (see WO 2004/061104).

Monoclonal Antibody Production. Monoclonal antibodies may be prepared using any technique which provides for the production of antibody molecules by continuous cell lines in culture. Such techniques include, but are not limited to, the hybridoma technique (see, e.g., Kohler & Milstein, Nature 256: 495-497 (1975)); the trioma technique; the human B-cell hybridoma technique (see, e.g., Kozbor, et al., Immunol. Today 4: 72 (1983)) and the EBV hybridoma technique to produce human monoclonal antibodies (see, e.g., Cole, et al., in: MONOCLONAL ANTIBODIES AND CANCER THERAPY, Alan R. Liss, Inc., pp. 77-96 (1985)). Human monoclonal antibodies can be utilized in the practice of the present technology and can be produced by using human hybridomas (see, e.g., Cote, et al., Proc. Natl. Acad. Sci. 80: 2026-2030 (1983)) or by transforming human B-cells with Epstein Barr Virus in vitro (see, e.g., Cole, et al., in: MONOCLONAL ANTIBODIES AND CANCER THERAPY, Alan R. Liss, Inc., pp. 77-96 (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the polypeptide. Alternatively, hybridomas expressing monoclonal antibodies can be prepared by immunizing a subject, e.g., with an isolated polypeptide comprising, or alternatively consisting essentially of, or yet further consisting of, the amino acid sequence of or a fragment thereof, and then isolating hybridomas from the subject's spleen using routine methods. See, e.g., Milstein et al., (Galfre and Milstein, Methods Enzymol 73: 3-46 (1981)). Screening the hybridomas using standard methods will produce monoclonal antibodies of varying specificity (i.e., for different epitopes) and affinity. A selected monoclonal antibody with the desired properties, e.g., can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the monoclonal antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981).

Phage Display Technique. As noted above, the antibodies of the present disclosure can be produced through the application of recombinant DNA and phage display technology. For example, antibodies, can be prepared using various phage display methods known in the art. In phage display methods, functional antibody domains are displayed on the surface of a phage particle which carries polynucleotide sequences encoding them. Phage with a desired binding property is selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fᵥ or disulfide stabilized Fᵥ antibody domains are recombinantly fused to either the phage gene III or gene VIII protein. In addition, methods can be adapted for the construction of Fab expression libraries (see, e.g., Huse, et al., Science 246: 1275-1281, 1989) to allow rapid and effective identification of monoclonal Fab fragments with the desired specificity for a polypeptide, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85: 5879-5883 (1988); Chaudhary et al., Proc. Natl. Acad. Sci. U.S.A., 87: 1066-1070 (1990); Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869 (1992); Sawai et al., AJRI 34: 26-34 (1995); and Better et al., Science 240: 1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g. Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Alternate Methods of Antibody Production. Antibodies may also be produced by inducing in vivo production in the lymphocyte population or by screening recombinant immunoglobulin libraries or panels of highly specific binding reagents (Orlandi et al., PNAS 86: 3833-3837 (1989); Winter, G. et al., Nature, 349: 293-299 (1991)).

Alternatively, techniques for the production of single chain antibodies may be used. Single chain antibodies (scFᵥs) comprise a heavy chain variable region and a light chain variable region connected with a linker peptide (typically around 5 to 25 amino acids in length). In the scFᵥ, the variable regions of the heavy chain and the light chain may be derived from the same antibody or different antibodies. scFᵥs may be synthesized using recombinant techniques, for example by expression of a vector encoding the scFᵥ in a host organism such as E. coli. DNA encoding scFᵥ can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFᵥ and a host transformed by the expression vector can be obtained according to conventional methods known in the art.

Antigen binding fragments can also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science, 256: 1275-1281 (1989)).

Antibody Modifications. The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFᵥ molecules, binding to streptavidin, introduction of a helix-turn-helix motif and the like can be exemplified.

The antibody compositions disclosed herein may be in the form of a conjugate formed between any of these antibodies and another agent (immunoconjugate). In one aspect, the antibodies disclosed herein are conjugated to radioactive material. In another aspect, the antibodies disclosed herein can be bound to various types of molecules such as polyethylene glycol (PEG).

Antibody Screening. Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between an antigenic fragment of HuD, e.g., anti-ELAVL4₁₋₁₁₇ or any fragment or oligopeptide thereof and its specific antibody. A two-site, monoclonal-based immunoassay utilizing monoclonal antibodies specific to two non-interfering epitopes may be used, but a competitive binding assay may also be employed (Maddox et al., J. Exp. Med., 158: 1211-1216 (1983)).

Antibody Purification. The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

### Methods of Use

General. The antibodies disclosed herein are useful in methods known in the art relating to the localization and/or quantitation of an antigenic fragment of HuD, e.g., ELAVL4₁₋₁₁₇ or an equivalent thereof. The antibodies disclosed herein are useful in isolating a HuD polypeptide by standard techniques, such as affinity chromatography or immunoprecipitation. An anti HuD, e.g., ELAVL4₁₋₁₁₇ or an equivalent of an antibody disclosed herein can facilitate the purification of natural HuD polypeptides from biological samples, e.g., mammalian sera or cells as well as recombinantly-produced HuD polypeptides expressed in a host system. Moreover, the antibody can be used to detect the polypeptide (e.g., in plasma, a cellular lysate or cell supernatant) in order to evaluate the abundance and pattern of expression of the polypeptide. The antibodies disclosed herein can be used diagnostically to monitor expression in tissue as part of a clinical testing procedure, e.g., to determine the efficacy of a given treatment regimen. The detection can be facilitated by coupling (i.e., physically linking) the antibodies disclosed herein to a detectable substance.

In another aspect, provided herein is a composition comprising an antibody or antigen binding fragment as disclosed herein bound to a peptide comprising, for example, a human HuD protein or a fragment thereof. In one aspect, the peptide is associated with a cell. For example, the composition may comprise a disaggregated cell sample labeled with an antibody or antibody fragment as disclosed herein, which composition is useful in, for example, affinity chromatography methods for isolating cells or for flow cytometry-based cellular analysis or cell sorting. As another example, the composition may comprise a fixed tissue sample or cell smear labeled with an antibody or antibody fragment as disclosed herein, which composition is useful in, for example, immunohistochemistry or cytology analysis. In another aspect, the antibody or the antibody fragment is bound to a solid support, which is useful in, for example: ELISAs; affinity chromatography or immunoprecipitation methods for isolating HuD proteins or fragments thereof, HuD positive cells, or complexes containing HuD and other cellular components. In another aspect, the peptide is bound to a solid support. For example, the peptide may be bound to the solid support via a secondary antibody specific for the peptide, which is useful in, for example, sandwich ELISAs. As another example, the peptide may be bound to a chromatography column, which is useful in, for example, isolation or purification of antibodies according to the present technology. In another aspect, the peptide is disposed in a solution, such as a lysis solution or a solution containing a sub-cellular fraction of a fractionated cell, which is useful in, for example, ELISAs and affinity chromatography or immunoprecipitation methods of isolating HuD proteins or fragments thereof or complexes containing HuD and other cellular components. In another aspect, the peptide is associated with a matrix, such as, for example, a gel electrophoresis gel or a matrix commonly used for western blotting (such as membranes made of nitrocellulose or polyvinylidene difluoride), which compositions are useful for electrophoretic and/or immunoblotting techniques, such as Western blotting.

Detection of HuD Polypeptide or Cell Expressing HuD. An exemplary method for detecting the level of HuD polypeptides or cells expressing HuD in a biological sample involves obtaining a biological sample from a subject and contacting the biological sample with an antibody disclosed herein which is capable of detecting the HuD polypeptides. Therapy can be monitored by detecting cells expressing HuD before and after treatment.

In one aspect, the antibodies or fragments thereof are detectably labeled. The term “labeled”, with regard to the antibody is intended to encompass direct labeling of the antibody by coupling (i.e., physically linking) a detectable substance to the antibody, as well as indirect labeling of the antibody by reactivity with another compound that is directly labeled. Non-limiting examples of indirect labeling include detection of a primary antibody using a fluorescently-labeled secondary antibody and end-labeling of a DNA probe with biotin such that it can be detected with fluorescently-labeled streptavidin.

The detection method of the present disclosure can be used to detect expression levels of HuD polypeptides in a biological sample in vitro as well as in vivo. In vitro techniques for detection of HuD polypeptides include enzyme linked immunosorbent assays (ELISAs), Western blots, flow cytometry, immunoprecipitations, radioimmunoassay, and immunofluorescence (e.g., IHC). Furthermore, in vivo techniques for detection of polypeptides include introducing into a subject a labeled anti-HuD antibody. By way of example only, the antibody can be labeled with a radioactive marker whose presence and location in a subject can be detected by standard imaging techniques. In one aspect, the biological sample contains polypeptide molecules from the test subject.

Immunoassay and Imaging. An antibody disclosed herein can be used to assay HuD polypeptide levels in a biological sample (e.g. human plasma) using antibody-based techniques. For example, protein expression in tissues can be studied with classical immunohistochemical (IHC) staining methods. Jalkanen, M. et al., J. Cell. Biol. 101: 976-985 (1985); Jalkanen, M. et al., J. Cell. Biol. 105: 3087-3096 (1987). Other antibody-based methods useful for detecting protein gene expression include immunoassays, such as the enzyme linked immunosorbent assay (ELISA) and the radioimmunoassay (MA). Suitable antibody assay labels are known in the art and include enzyme labels, such as, glucose oxidase, and radioisotopes or other radioactive agents, such as iodine (¹²⁵I, ¹²¹I, ¹³¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying HuD polypeptide levels in a biological sample, HuD polypeptide levels can also be detected in vivo by imaging. Labels that can be incorporated with anti-HuD antibodies for in vivo imaging of HuD polypeptide levels include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the HuD antibody by labeling of nutrients for the relevant scFᵥ clone.

An HuD antibody which has been labeled with an appropriate detectable imaging moiety, such as a radioisotope (e.g.,131I, ¹¹²In, ⁹⁹mTc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled HuD antibody will then preferentially accumulate at the location of cells which contain the specific target polypeptide. For example, in vivo tumor imaging is described in S. W. Burchiel et al., Tumor Imaging: The Radiochemical Detection of Cancer 13 (1982).

In some aspects, HuD antibodies containing structural modifications that facilitate rapid binding and cell uptake and/or slow release are useful in in vivo imaging detection methods. In some aspects, the HuD antibody contains a deletion in the CH2 constant heavy chain region of the antibody to facilitate rapid binding and cell uptake and/or slow release. In some aspects, a Fab fragment is used to facilitate rapid binding and cell uptake and/or slow release. In some aspects, a F(ab)′2 fragment is used to facilitate rapid binding and cell uptake and/or slow release.

Diagnostic Uses of HuD antibodies. The HuD antibody compositions disclosed herein are useful in diagnostic and prognostic methods. As such, the present disclosure provides methods for using the antibodies disclosed herein in the diagnosis of medical conditions as disclosed herein in a subject. Antibodies disclosed herein may be selected such that they have a high level of epitope binding specificity and high binding affinity to the HuD polypeptide. In general, the higher the binding affinity of an antibody, the more stringent wash conditions can be performed in an immunoassay to remove nonspecifically bound material without removing the target polypeptide. Accordingly, HuD antibodies of the present technology useful in diagnostic assays usually have binding affinities of at least 10⁻⁶, 10⁻⁷, 10⁻⁸, 10⁻⁹, 10⁻¹⁰, 10⁻¹¹, or 10⁻¹² M. In certain aspects, HuD antibodies used as diagnostic reagents have a sufficient kinetic on-rate to reach equilibrium under standard conditions in at least 12 hours, at least 5 hours, at least 1 hour, or at least 30 minutes.

Some methods of the present technology employ polyclonal preparations of anti-HuD antibodies and polyclonal anti-HuD antibody compositions as diagnostic reagents, and other methods employ monoclonal isolates. In methods employing polyclonal human anti-HuD antibodies prepared in accordance with the methods described above, the preparation typically contains an assortment of HuD antibodies, e.g., antibodies, with different epitope specificities to the target polypeptide. The monoclonal anti-HuD antibodies of the present disclosure are useful for detecting a single antigen in the presence or potential presence of closely related antigens.

The HuD antibodies of the present disclosure can be used as diagnostic reagents for any kind of biological sample. In one aspect, the HuD antibodies disclosed herein are useful as diagnostic reagents for human biological samples. HuD antibodies can be used to detect HuD polypeptides in a variety of standard assay formats. Such formats include immunoprecipitation, Western blotting, ELISA, radioimmunoassay, flow cytometry, IHC and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue (including biopsies), cell or body fluid of a subject.

Prognostic Uses of Antibodies. The present disclosure also provides for prognostic (or predictive) assays for determining whether a subject is at risk of developing a medical disease or condition associated with increased polypeptide expression or activity (e.g., detection of a precancerous cell). Such assays can be used for prognostic or predictive purpose to thereby prophylactically treat an individual prior to the onset of a medical disease or condition characterized by or associated with polypeptide expression.

Another aspect of the present disclosure provides methods for determining HuD expression in a subject to thereby select appropriate therapeutic or prophylactic compounds for that subject.

### Therapeutic Methods

Also provided herein is a method of treating a cancer in a subject in need thereof, comprising, or alternatively consisting essentially of, or yet further consisting of administering to the subject an effective amount of an antibody or antigen or antigen binding fragment that binds specifically to an isoaspartylated protein or a fragment thereof. In one aspect, the the cancer is a solid tumor. In another aspect, the cancer is a cancer in tissues that would not normally express neuronal proteins. Non-limiting examples of such include a cancer selected from small cell lung cancer (SCLC), breast cancer, melanoma, and a gynecological cancer.

The subject is an animal such as a sport animal, a pet or a farm animal. In another aspect, the subject is a human patient. One of skill in the art can determine when the treatment has been effective by assaying a sample isolated from the subject for antibodies that specifically recognize and bind the protein or fragment thereof, or by noting a cellular immune response.

In one aspect, the protein is an embryonic lethal altered visual system-like RNA-binding protein (Hu protein, optionally HuD protein). In another aspect, the protein comprises an isoaspartylated residue. In a further aspect, the fragment comprises an amino acid capable of isoAsp conversion. In a yet further aspect, the fragment comprises one or more isoaspartylated residue. In a yet further aspect, the fragment comprises an RNA recognition motif 1 (RRM1).

The therapy can be combined with other anti-cancer therapies which can be administered simultaneously or in sequence as determined by the treating physician or veterinarian in one or more doses over a course of treatment. The treatment is a first-line, second-line, third-line, fourth-line, fifth-line or subsequent treatment.

In one aspect, the antibody is a monoclonal antibody or a polyclonal antibody. In a further aspect, the antibody is humanized. In a further aspect the antigen binding fragment is derived from a humanized antibody.

### Kits

As set forth herein, the present disclosure provides therapeutic methods for treating cancer or inducing an immune response to a cancer as well as diagnostic methods for determining the expression level of a protein or polypeptide as described herein. In one particular aspect, the present disclosure provides kits for performing these methods as well as instructions for carrying out the methods of the present disclosure such as administration doses and protocol, collecting tissue and/or performing the screen, and/or analyzing the results.

The kit comprises, or alternatively consists essentially of, or yet further consists of, polypeptide or antibody composition (e.g., monoclonal antibodies) disclosed herein, and instructions for use. The kits are useful for in the therapeutic methods as described herein and for detecting the presence of polypeptides in a biological sample e.g., any body fluid including, but not limited to, e.g., sputum, serum, plasma, lymph, cystic fluid, urine, stool, cerebrospinal fluid, acitic fluid or blood and including biopsy samples of body tissue such as tumor. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

In some aspects, the kit can comprise: one or more polypeptides, proteins or antibodies of this disclosure that can be labeled. The kit components, (e.g., reagents) can be packaged in a suitable container. The kit can further comprise instructions for using the kit to for the intended purpose. In certain aspects, the kit comprises a first antibody, e.g., attached to a solid support, which binds to polypeptide; and, optionally; 2) a second, different antibody which binds to either the polypeptide or the first antibody and is conjugated to a detectable label.

The kit can also comprise compositions comprising a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### Carriers

The antibodies, polynucleotides, vectors, polypeptides, and/or host cells, also can be bound to many different carriers. Thus, this disclosure also provides compositions containing the antibodies and another substance, active or inert. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the disclosure. Those skilled in the art will know of other suitable carriers for binding antibodies, or will be able to ascertain such, using routine experimentation.

### Materials and Methods

Sera were collected from Dutch patients with PEM/SN and/or SCLC in the University Hospital of Maastricht, the Netherlands, and were obtained with informed consent and approval by the medical-ethical committees. Applicants used sera previously determined to be positive for anti-ELAVL4 (“anti-HuD”) reactivity to gain a better understanding of the nature of the immune response. Use of the human serum samples was approved by the USC Institutional Review Board (protocol #HS-10-00050).

### Recombinant Protein Production, In Vitro Isoaspartyl (IsoAsp) Conversion and Detection

Similar to the N-terminal fragment used to broadly characterize the ELAVL immune epitopes in numerous studies (Manley et al., 1995; Sillevis Smit et al., 1996; Sodeyama et al., 1999; Graus et al., 1998), Applicants produced recombinant human ELAVL4 (aa 1-117) polypeptide with a C-terminal hexahistidine tag. Recombinant ELAVL4₁₋₁₁₇ was made in BL21 (DE3) E. coli carrying a plasmid providing extra copies of a rare Arginine tRNA. Protein production was very high, so that high purity was achieved. In addition, all constructs carry the full RRM, forming a globular domain that folds well and that at purification is present in very high concentrations in solution. Applicants measured endotoxin levels using the Pierce LAL Chromogenic Endotoxin Quantitation Kit (catalogue number 88282, Thermo Scientific, Rockford, Ill.) and levels were found to be negligible or undetectable. Applicants used protein batches with undetectable endotoxin levels for experiments with human peripheral blood monocytes. ELAVL4 mutants were generated by site-directed mutagenesis and verified by sequencing. The mouse and human ELAVL4 N-terminal regions (aa 1-117) are identical except for a single conservative change (threonine 33 in human to alanine in mouse). Recombinant DNA work was carried out under BSL1 containment conditions. Proteins were incubated in 50 mM K-HEPES (pH 7.4), 1.0 mM EGTA, 0.02% (w/v) sodium azide, and 5% (w/v) glycerol) for up to 7 days at 37° C., 1 μg per lane was resolved on protein gels and transferred to membranes for on-blot methylation with recombinant rat repair enzyme protein-L-isoaspartate O-methyltransferase (PIMT) and [³H]—S-adenosyl methionine (Zhu et al., 2006).

### Anti-IsoAsp ELAVL4 Antiserum Generation and Western Blotting

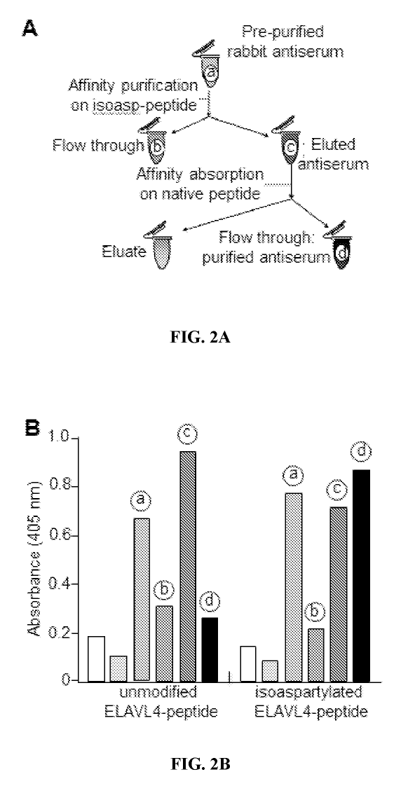

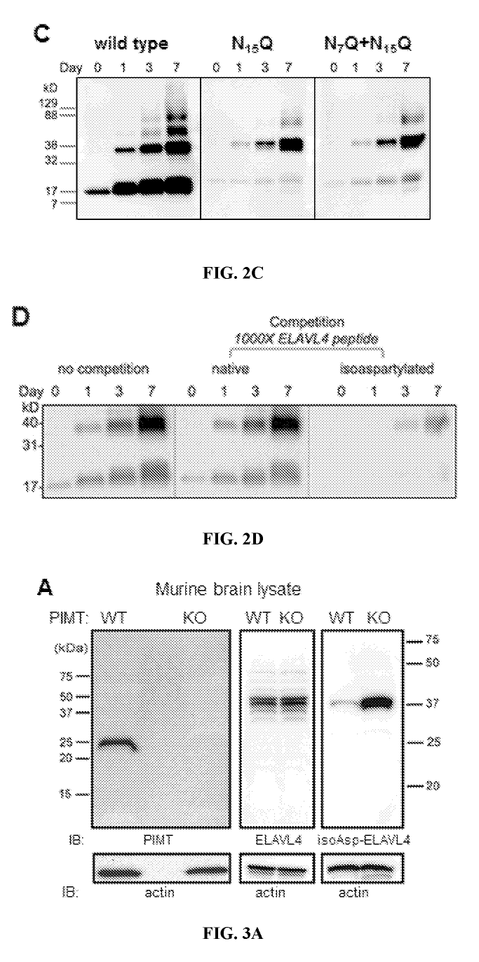

Rabbits were immunized with synthetically generated CTSNTS-isoAsp-GPSSNNR-amide peptide, conjugated to a carrier protein (YenZym Antibodies, San Francisco, Calif.). The antibody was affinity-purified using the isoAsp-containing peptide, then affinity-absorbed against the unmodified peptide. Specificity was analyzed by ELISA (YenZym Antibodies, see FIGS. 2A-2B). The affinity-purified anti-isoAsp-ELAVL4 antibody (1:250) was used with secondary goat anti-rabbit IgG HRP conjugate (1:20,000) to detect isoaspartylation on western blots containing 0.25-1 μg recombinant proteins, as indicated in the figure legends. For the peptide competition assay, 1 μg/m1 of antibody was pre-incubated with 500-1000-fold molar excess of the native or isoAsp-containing ELAVL4 peptide at 4° C. overnight. For detection of isoAsp conversion in brain lysates, ˜50 μg of brain extract from PIMT wild type and knockout mice was subjected to western blot analysis with primary antibodies: rabbit anti-isoAsp ELAVL4 (1:250); rabbit anti-actin (1:250, Cytoskeleton Inc., Denver, Colo.); mouse anti-human PIMT (1:1000, Santa Cruz Biotechnology, CA); rabbit anti-human ELAVL4 (1:250, Santa Cruz Biotechnology, CA). Secondary antibodies were added for one hour at room temperature with gentle agitation, either goat anti-rabbit IgG (1:20,000) or goat anti-mouse IgG (1:2000). Restore Plus Western Blot Stripping Buffer was used to strip and reprobe blots according to manufacturer's instructions (Thermo Scientific, Rockford, Ill.).

### Immunohistochemistry

Serial 4 μm thick sections of de-identified archival remnants of paraffin-embedded human SCLC tumor samples (National Disease Research Interchange, Philadelphia, Pa.) were subjected to haematoxylin/eosin staining or incubation with primary antibodies: rabbit anti-human ELAVL4 1:250 (Santa Cruz Biotechnology, Santa Cruz, Calif.); Applicants' rabbit anti-isoAsp ELAVL4 serum 1:400 (0.625 μg/m1 final concentration; YenZym, South San Francisco, Calif.); rabbit anti-human PIMT 1:1000 (Santa Cruz Biotechnology, Santa Cruz, Calif.). The EnVision+System-HRP labeled polymer anti-rabbit (K4002, Dako North America, Inc., Carpinteria, Calif.) was visualized with liquid DAB chromogen (Biogenex, Fremont, Calif.), and ethyl-green applied as counter staining. Images were captured using the Aperio imaging system and processed using the compatible ImageScope viewing software. Sections were digitally analyzed in the range of 2× to 40× magnification.

### Mouse and Human Immunogenicity Assays

FVB/N mice were immunized subcutaneously with 150 μg recombinant protein emulsified 1:1 with incomplete Freund's adjuvant and boosted at 7 days; spleens were harvested 10 days later. Blood was collected before immunization, just prior to boosting and at euthanasia. Plasma was tested for reactivity to native and isoAsp ELAVL4 using goat-anti-mouse IgG-HRP secondary antibody (1:2000). Due to very limited amounts of mouse blood, Applicants first tested several different dilutions of the mouse plasma from preimmunized, pre-boosted and terminal bleed mice to determine which resulted in signal in the linear range. Applicants then used the same dilution to tests all pre-immunization sera (1:2000), pre-boosting sera (1:2000) and terminal bleed plasma samples (1:40,000), using identical exposure times for all blots. Applicants quantitated the blots using Bio-Rad imaging software, drawing a box around the bands in each lane and subtracting background activity for a similar size box in an area without signal. For mouse T-cell proliferation assays, single cell suspensions were prepared from harvested spleens. 300,000 cells were seeded in triplicate into pre-titrated 96-well culture plates containing serial dilutions of native or isoAsp-converted ELAVL4 protein, vehicle, or positive control. Cells were pulsed with 0.5 mCi [³H] thymidine/well at 72 hours, incubated for 18 hours, harvested and counted using a TopCount Scintillation Counter (Perkin Elmer, Foster City, Calif.). Thymidine uptake in counts per minute (cpm) was determined by averaging replicate wells. All animal experiments were approved by the USC institutional animal care and use committee. Human peripheral blood mononuclear cells from unidentified donors were incubated for 3 days with 1 μg/mL native or isoAsp-ELAVL4₁₋₁₁₇, then pulsed with [³H]thymidine, incubated for 24 h, harvested and counted.

ELAVL4 Serum Reactivity from Anti ELAVL4 Positive SCLC-Patients

Serum samples from seven patients previously shown to exhibit an anti-ELAVL4 response were examined at dilutions ranging from 1:1000 to 1:10,000 depending on previously observed anti-ELAVL4 reactivity. Secondary antibody was HRP-conjugated anti-human IgG (1:2,000).

### Statistical Methods

The student's two-sided t-test was used with a significance value of p<0.05.

### In Vitro Isoaspartyl Conversion of ELAVL4

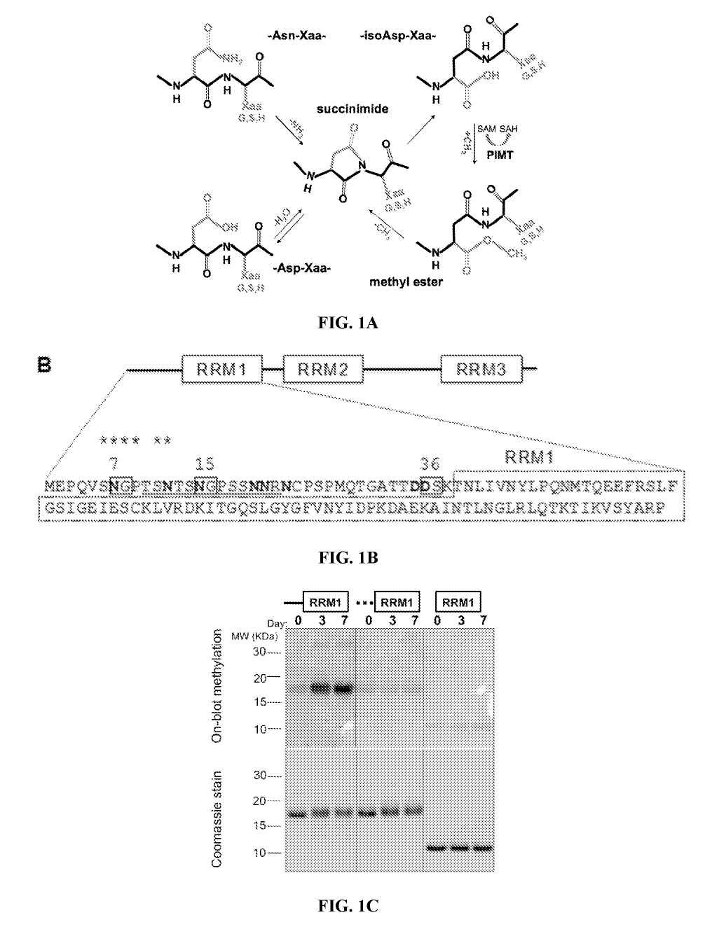

In vitro incubation under physiological conditions can be used to determine if a protein is highly susceptible to isoaspartylation (Johnson et al., 1989) (FIG. 1A). Recombinant human ELAVL4 was incubated at pH 7.4 and 37° C. for up to seven days. Initially, the experiment was performed with full-length ELAVL4, which contains four potential canonical isoAsp-prone sites (amino acids D or N followed by G, S, or H in flexible regions of the protein). However, this protein quickly precipitated under the experimental conditions and was not used for any further experiments (data not shown). Because the N-terminal domain of ELAVL4 containing RNA recognition motif 1 (RRM1) had been strongly implicated in immune responses in SCLC patients (Manley et al., 1995; Sillevis Smit et al., 1996; Sodeyama et al., 1999; Graus et al., 1998), Applicants proceeded with this segment of the protein (amino acids 1-117). The region N-terminal to RRM1 contains three potential canonical isoAsp conversion sites (N7, N15, and D36) and five additional N or D residues (FIG. 1B). Recombinant human ELAVL4₁₋₁₁₇ was incubated for 0, 1, 3, and 7 days and tested for the presence of isoaspartylation by its ability to accept a radiolabeled methyl group (3H-S-adenosyl methionine) in an on-blot PIMT-catalyzed repair reaction (FIG. 1C). Native ELAVL4₁₋₁₁₇ exhibited little reactivity (Day 0), but the protein showed a strong increase in PIMT-mediated methylation over the incubation period. Mutation of the canonical isoAsp-prone sites N7, N15 and D36 only reduced labeling by about 25% (not shown), indicating that additional sites are present. A mutant polypeptide fragment in which all eight N or D residues in the 38-aa N-terminal region had been mutated to Q or E (residues that are similar but not prone to isoaspartylation), did not appear to become isoaspartylated, nor did RRM1 alone (FIG. 1C). Thus Applicants conclude that the region N-terminal to RRM1 becomes isoaspartylated in vitro under conditions of physiological pH and temperature.

### In Vivo Isoaspartylation of ELAVL4

To determine whether isoaspartylation occurs in vivo, Applicants raised a rabbit anti-isoAsp ELAVL4 antiserum. The region around the canonical isoaspartylation site N₁₅ was determined to be a strong candidate for peptide generation based on its predicted antigenicity, hydrophilicity, secondary structure, and potential for post-translational events. Rabbits were immunized with a peptide containing an isoAsp residue at N₁₅ and an N-terminal cysteine: CTSNTS-isoAsp-GPSSNNR (FIG. 1B, underlined). Serum was collected and affinity purified on a column carrying the isoAsp-containing peptide, followed by affinity absorption of non-isoAsp-specific reactivity on a native peptide column (FIG. 2A). Whereas the antibody purified from the first column recognized both the native and isoAsp forms of the peptide, following immuno-absorption, the antibody showed considerable specificity for the isoAsp-containing ELAVL4 peptide as demonstrated by ELISA (FIG. 2B). To verify the ability of the affinity-purified/absorbed antibody to specifically recognize isoAsp-containing ELAVL4, Applicants examined its reactivity against ELAVL4₁₋₁₁₇, a single N₁₅Q mutant and an N₇Q+N₁₅Q double mutant, all incubated under isoaspartylation-inducing conditions as in FIG. 1C. The double mutant was generated because the protein sequence in the N₁₅ region is partially homologous to the region containing N₇, and the antiserum could therefore be cross-reactive with this region (see FIG. 1B, starred). Western blot analysis using the purified anti-isoAsp ELAVL4 antiserum showed some reactivity to wild type protein at day 0, possibly due to residual cross reactivity of the antiserum with the unconverted peptides or to limited isoAsp conversion during protein production (FIG. 2C). However, over time, ELAVL4₁₋₁₁₇ showed a dramatic increase in reactivity. Additionally, during the incubation period reactivity appeared against bands of higher molecular weight, which seem to be multimers of the protein that are not disaggregated despite denaturing conditions. Isoaspartylation has been implicated in multimerization and aggregation (Shimizu et al., 2000; Kern et al., 2005; Zirah et al., 2006; Paranandi and Aswad, 1995). The ELAVL4₁₋₁₁₇ multimers are barely visible on Coomassie gels but do become visible during the on-blot methylation (FIG. 1C), suggesting that they contain isoAsp sites and are substrates for repair by PIMT.

The N₁₅Q and N₇Q+N₁₅Q mutant proteins showed weaker reactivity with the rabbit antiserum than the wild type protein, suggesting that the antiserum is specific for the N₁₅ site (FIG. 2C). However, some reactivity accumulated over time and the higher molecular weight complex still showed substantial signal, suggesting that the aggregate is highly reactive with the antibodies. To further examine the isoAsp specificity of the antibody, Applicants incubated the antiserum with a 1000-fold molar excess of either the native or isoAsp-containing ELAVL4 peptide (FIG. 2D). While the native peptide did not compete, the isoAsp peptide almost completely prevented recognition of in vitro isoAsp-converted ELAVL4₁₋₁₁₇ protein, confirming ELISA data indicating that the antiserum shows considerable specificity for the post-translational modification. Applicants also tested the antiserum for cross reactivity with the other ELAVL family proteins and observed reactivity with the two other neuronal ELAVL family members (Pulido et al, 2016). The highly conserved ELAVL2 (He1N1/HuB) showed a strong interaction and ELAVL3 (HuC) a weaker one. In contrast, the antisera did not react with the ubiquitously expressed ELAVL1 (HuR).

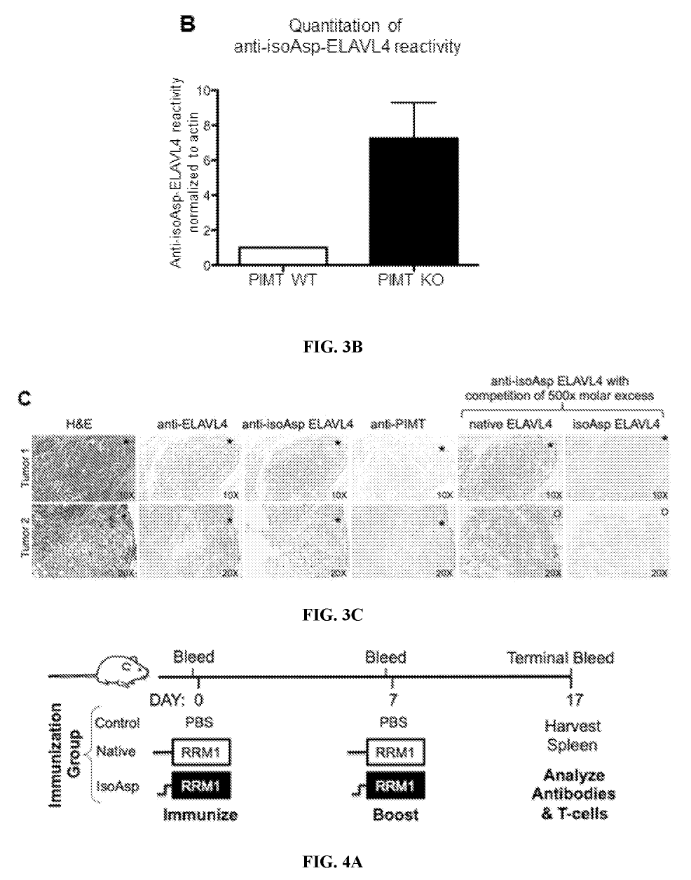

To determine whether isoaspartylation of neuronal ELAVL proteins occurs in vivo, Applicants used the affinity purified anti-isoAsp-ELAVL4 rabbit antiserum to examine brain lysates from PIMT knockout mice, which are known to accumulate high levels of isoAsp-containing proteins in the brain, a place where PIMT is highly expressed (Zhu et al., 2006; Kim et al., 1997; Yamamoto et al., 1998). The isoAsp-ELAVL4-specific serum was much more reactive with lysates from PIMT knockout mice than control animals (FIGS. 3A-3B), indicating that neuronal ELAVL proteins can undergo isoaspartylation in vivo.

Applicants next used the anti-isoAsp-ELAVL4 rabbit antiserum to examine human SCLC tumors; neuronal ELAVL proteins are among many abnormally expressed neuronal proteins observed in SCLC tumors (Manley et al., 1995) and might accumulate isoaspartylation due to ineffective repair of spontaneously isoaspartylated protein. Because SCLC patients are rarely operated, paraffin-embedded tumor samples are rare (most diagnoses are made by fine needle aspiration). Applicants obtained and analyzed 5 paraffin-embedded tumors; as expected all showed high anti-ELAVL4 reactivity (Manley et al., 1995; Dalmau et al., 1992) as well as reactivity with the anti-isoAsp-ELAVL4 antiserum (FIG. 3C, and data not shown). Competition with ELAVL4₁₋₁₁₇ incubated under isoaspartylation conditions but not native ELAVL4₁₋₁₁₇ depleted reactivity with the isoAsp-specific antiserum, supporting the specificity of the assay. Probing of adjacent sections with an antiserum to PIMT showed little staining. PIMT is most highly expressed in the nervous system and is normally present but low in the lung. Applicants' results strongly support the presence of neuronal isoAsp-ELAVL proteins in SCLC.

### Immunogenicity of Isoaspartylated ELAVL4₁₋₁₁₇

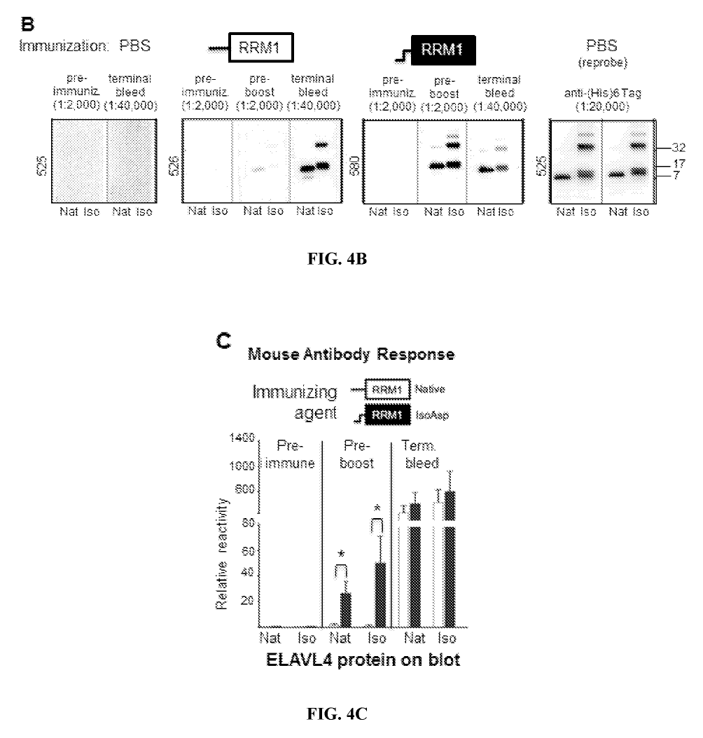

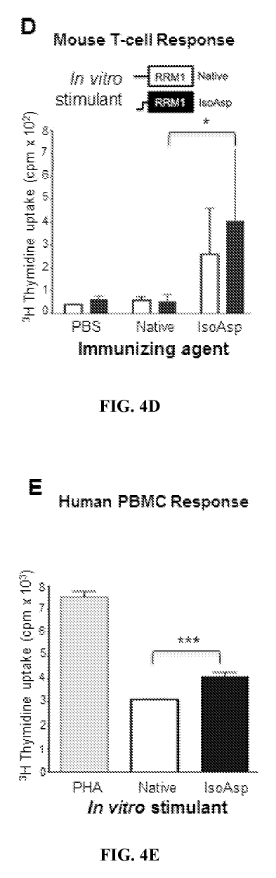

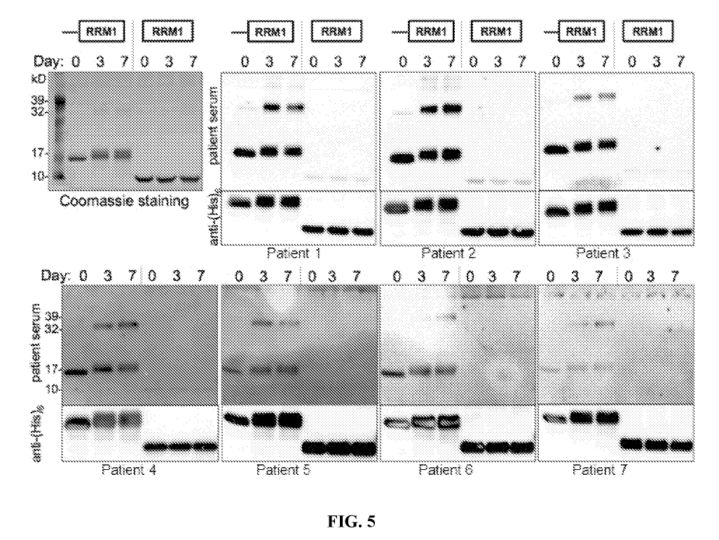

To test the effect of isoaspartylation on the immunogenicity of the ELAVL4₁₋₁₁₇ fragment, Applicants immunized mice subcutaneously with the native ELAVL4₁₋₁₁₇ fragment or protein that had been incubated under isoAsp-inducing conditions for 7 days. Mice were boosted after one week and euthanized 10 days later. B- and T-cell responses were evaluated (FIG. 4A). Mice immunized with isoAsp-converted protein showed an early and strong antibody response even without a booster injection (FIGS. 4B-4C). As expected based on previous immunization experiments with isoAsp-containing peptides (Mamula et al., 1999), antisera reacted with both native and isoaspartylated protein. IsoAsp-ELAVL4₁₋₁₁₇ immunized mice also showed a stronger T-cell response than mice that had been immunized with native ELAVL4₁₋₁₁₇ (FIG. 4D).

To corroborate these results on human immune cells, Applicants incubated human peripheral blood mononuclear cells (PBMC) with native or isoaspartylated ELAVL4₁₋₁₁₇, pulsed them with [³H]thymidine after three days, and measured cell proliferation 1 day later. Cell proliferation in PBMC treated with isoAsp-ELAVL4₁₋₁₁₇increased significantly compared to PBMC treated with native ELAVL4₁₋₁₁₇(FIG. 4E). Thus, as in the mouse, human immune cells appear to respond more strongly to isoaspartylated ELAVL4₁₋₁₁₇, supporting the idea that unrepaired isoAsp-ELAVL4 could trigger immune responses in SCLC patients. In all assays, the difference in immune response to native vs. isoAsp protein was likely underestimated because the native protein undergoes spontaneous isoaspartylation during the immunization/incubation periods.

Sera from Anti-ELAVL-Positive SCLC Patients React with the IsoAsp Prone N-Terminal Region

If isoaspartylation causes anti-ELAVL reactivity in SCLC, and without being bound by theory, it was hypothesized that reactivity against the N-terminal isoAsp-prone region of ELAVL4 should be detected using patient antisera. Applicants examined 7 antisera from patients that had been previously confirmed to show anti-ELAVL activity. Three sera were from patients with the anti-ELAVL (“anti-Hu”) syndrome PEM/SN and neurological symptoms, one from a SCLC patient with anti-ELAVL response but a different paraneoplastic disease, Lambert Eaton myasthenic syndrome (LEMS), and three from SCLC patients with a positive but weaker immune response and no neurological symptoms (Table 1). The latter type of patients is much more common than patients with high titer antibodies (Graus et al., 1997; Kazarian and Laird-Offringa, 2011; Dalmau et al., 1990).

TABLE 1
Properties of anti-ELAVL-positive patients¹

| | Paraneoplastic | | Age at | Smoking | Tumor and | Age | Survival | Initial response to |
|---|---|---|---|---|---|---|---|---|
| Patient | Disease | Gender | diagnosis | History | stage | deceased | (months) | treatment |
| 1 | PEM/SN | F | 72 | Unknown | SCLC, | 72 | 0 | No treatment |
| | | | | | LD | | | |
| 2 | PEM/SN | F | 66 | 50 pack- | n/a; no | 76 | n/a | n/a |
| | | | | years | SCLC or | | | |
| | | | | | other | | | |
| | | | | | tumor | | | |
| 3 | PEM/SN | F | 43 | Unknown | Primary | 45 | 21 | Complete |
| | | | | | unclear, | | | response |
| | | | | | ED | | | |
| 4 | LEMS | M | 76 | 75 pack- | SCLC, | 84 | 99 | Complete |
| | | | | years | LD | | | response |
| 5 | none | F | 65 | Unknown | SCLC, | 66 | 10 | Complete |
| | | | | | | | | response |
| 6 | none | M | 74 | Unknown | SCLC, | 74 | 0 | No response |
| | | | | | LD | | | |
| 7 | none | F | 55 | Previous | SCLC, | 56 | 8 | No response |
| | | | | smoker | LD | | | |

LD: limited disease,
ED: extensive disease.

Patients 1-3 were diagnosed with PEM/SN, which is typically associated with anti-ELAVL antibodies; patient 2 showed no detectable tumor in 11-year follow up despite suspected SCLC; patient 3 showed neuroendocrine brain metastasis but no primary tumor at the time -origin of metastasis was unclear since she had a history of ovarian carcinoma and pulmonary metastasis; patient 4 showed anti-ELAVL4 antibodies but exhibited LEMS, which is associated with antibodies against voltage-gated calcium channels; patients 5-7 showed no paraneoplastic autoimmune neurological disease.

Sera were tested by western blot for reactivity with ELAVL4₁₋₁₁₇ or RRM1 alone (Applicants were unable to produce ELAVL4₁₋₃₈ because it precipitated), both incubated under isoaspartylation-inducing conditions (FIG. 5). As with the rabbit antiserum, the human sera reacted strongly with isoaspartylated protein and higher molecular weight complexes of ELAVL4₁₋₁₁₇. Sera from SCLC patients previously shown to be negative for anti-ELAVL4 reactivity lacked reactivity with both native and isoAsp-ELAVL4. It is remarkable that rabbits immunized with a short synthetic isoaspartylated ELAVL4 peptide generated an immune response so similar to that of human SCLC patients, whose exposure to ELAVL4 is linked to the aberrant expression of the protein in their tumor. This further supports the notion that the patient antigenic response is linked to isoaspartylation. The human sera also reacted with native protein (Day 0), a phenomenon frequently observed when isoAsp-containing peptides are used to immunize animals, and attributed to epitope spreading (Mamula et al., 1999; Mamula, 1998). Notably, all sera Applicants tested from patients with moderate to strong ELAVL4 reactivity showed weak or no reactivity with the RRM1 domain alone, indicating that the epitope(s) against which the patient antibodies are directed are located in the 38-amino acid N-terminal region (FIG. 5, and data not shown).

For over two decades it has remained unknown why SCLC patients exhibit unusual immune responses to neuronal proteins that are abnormally expressed in their tumors. Here, Applicants report the results of a study to understand the anti-ELAVL4 response to solve this enigma. Previous experiments using recombinant protein and deletion mutants using numerous SCLC patient antisera known to be anti-ELAVL positive have consistently pointed to the N-terminal portion of the protein which carries RRM1 (Manley et al., 1995; Sillevis Smit et al., 1996; Sodeyama et al., 1999; Graus et al., 1998). This 117-amino acid N-terminal protein fragment showed reactivity with every patient serum sample tested. The identical region was identified using sera from 7 transgenic mice with SCLC that were anti-ELAVL4 positive (Kazarian et al., 2009). However, none of these experiments examined RRM1 in isolation. Applicants consistently observed that reactivity against RRM1 alone was much weaker or absent in sera from SCLC patients despite an antibody response to the full 117-amino acid fragment. This suggests that the 38-amino acid region N-terminal to RRM1 plays a key role in the SCLC-associated autoimmune response. A previous epitope mapping experiment using the N-terminal amino acids fused to a partial RRM1 did not detect reactivity with SCLC patient antisera (Sodeyama et al., 1999). However, the authors did not demonstrate successful purification of the recombinant protein fragment and Applicants have found that effective production of the N-terminal piece requires the globular RRM, possibly as a solubility “handle”. Combined with our demonstration that ELAVL4 becomes isoaspartylated in vitro and in vivo, the well-documented immunogenicity of isoaspartylated proteins and peptides (Yang et al., 2006; Doyle et al., 2003), and Applicants' demonstrated immunogenicity of isoaspartylated ELAVL4₁₋₁₁₇, the data make a strong case for isoaspartylation of neuronal ELAVL proteins as the trigger for SCLC-associated anti-ELAVL reactivity.

Isoaspartylation happens naturally under physiological conditions and the repair enzyme PIMT is expressed in all organisms from bacteria to mammals. While PIMT is present in all mammalian tissues, it is most prominent in the brain, retina, and testis (Diliberto and Axelrod, 1976; Mizobuchi et al., 1994; Qin et al., 2014). PIMT is crucial for normal neuronal activity; mice lacking the enzyme show a dramatic increase in protein isoaspartylation in the brain and die of seizures a few weeks after birth (Kim et al., 1997; Yamamoto et al., 1998; Desrosiers and Fanelus, 2011). This would suggest that neuronal proteins, either through their function or location, are more prone to isoAsp conversion. Misexpression of neuronal proteins in non-neuronal types of cells, such as SCLC tumors, might lead to the accumulation of isoaspartylated proteins. PIMT levels in SCLC may not be as high as in the nervous system Applicants detected little if any reactivity with PIMT antibodies in SCLC tumors), and conditions such as stress, hypoxia and/or necrosis might favor isoaspartylation. It is also possible that the repair enzyme might not be able to access damaged protein spilling from necrotic cells. Thus, the amount of isoaspartylated protein to which SCLC patients are exposed might vary based on the level of expressed PIMT, the amount of necrosis, or other factors, such as single nucleotide polymorphisms that are known to affect PIMT efficacy (Qin et al., 2014; Desrosiers and Fanelus, 2011). Exposure to sufficient levels of isoaspartylated ELAVL proteins to trigger an immune response might be a stochastic event and may not happen in all SCLC patients, even if all SCLC tumors express neuronal ELAVL family members. Indeed, in an inbred transgenic SCLC mouse model, despite ubiquitous expression of neuronal ELAVL proteins in the cancers, only 14% of mice developed a detectable anti-ELAVL response (Kazarian et al., 2009).

Based on Applicants' data with ELAVL4, one would expect isoaspartylation to play a role in paraneoplastic responses to other SCLC-associated antigens. Well over 50 neuronal proteins (many as yet uncharacterized) undergo isoaspartylation in the brains and retinas of PIMT knockout mice (Zhu et al., 2006; Qin et al., 2014). Two of these, alpha-enolase and recoverin, are indeed antigens in SCLC-associated retinopathy (Adamus et al., 1996). Interestingly, the paraneoplastic retinopathy-associated antigens are not only misexpressed in SCLC, but also in breast cancer, melanoma and gynecological cancers, all tissues that would normally not express neuronal proteins (Adamus, 2009). Thus, the findings of Applicants' study may have implications for numerous types of cancer. Examination of the sequence and predicted structure of known cancer-associated antigens reveals many potential isoaspartylation sites. For example, cerebellar degeneration-related protein 2 (CDR2), targeted by characteristic “anti-Yo” antibodies mainly in ovarian cancer patients, carries numerous potential isoaspartylation sites in the regions linking the known protein domains. Just like ELAVL4 in SCLC, the CDR2 antigen was present in all tested ovarian tumors, but only a quarter of patients carry anti-Yo antibodies (Totland et al., 2011). Isoaspartylation of neuronal antigens might also occur in non-cancer patients. For example, in at least one dementia patient with anti-NMDA receptor autoantibodies, the antigenic site lay in a loop containing an NG sequence—a typical isoaspartylation-prone sequence—and is abolished by the N368Q mutation (Doss et al., 2014), which prevents isoaspartylation. Investigating the propensity of neuronal antigens to undergo isoaspartylation in SCLC as well as other disease states will be important, but may be challenging due to the tendency of proteins to aggregate or precipitate under isoaspartylation conditions. The antiserum Applicants raised was context-dependent and did not react with other isoaspartylated proteins (FIG. 2C and unpublished data). A generic isoAsp-reactive antibody would be very useful but it may be difficult to generate as the isoAsp epitope is shallow and embedded in the flanking protein sequence (Griffith et al., 2001).

Applicants' data show that isoaspartylation of ELAVL4 and other neuronal antigens is the trigger for SCLC-associated autoimmune responses. Isoaspartylated antigens could be powerful new cancer-specific targets for immunotherapy in SCLC. It was shown in a mouse model that isoAsp-conversion of melanoma antigen TRP-2 (tyrosinase-related protein-2) resulted in the generation of a T-cell response in vitro and in vivo in which CD8+ T cells were recruited to melanoma, and autoantibodies capable of binding tumor cells were produced (Doyle et al., 2006). Tumor growth was delayed upon isoAsp TRP-2 immunization, perhaps influencing immunologic clearance of the tumor. Indeed, anti-ELAVL4 autoantibodies in SCLC patients have been correlated with limited disease, complete response to therapy and improved survival (Graus et al., 1997) and have been associated with anecdotal spontaneous regression (Darnell and DeAngelis, 1993). Applicants' mouse immunization experiments show that strong B- and T-cell responses against ELAVL4 can be elicited with isoaspartylated protein and provide pre-clinical evidence for the feasibility of immunotherapy. It is intriguing that mice immunized with the native protein do not show a T-cell response but do develop antibodies after 17 days. One challenge of immunization experiments with native protein remains the possibility that proteins may undergo conversion and aggregation during the experiment. Since isoAsp moieties are known to aggregate, it is possible that the aggregates might form in the immunized location and might trigger a partially T-cell independent response, which might explain the lack of T-cell response seen in the case of the native protein. The structure of isoAsp aggregates is unknown and how such aggregates, not only of ELAVL4 but of other isoaspartylated proteins might play a role in directing the immune response is a very interesting question. The study of immune triggering by isoaspartylated proteins is still in its infancy.

### Equivalents

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scope of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. Throughout this disclosure, various publication are referenced by a citation, the full bibliographic citation for each are provided immediately preceding the claims.

Sequence Listing-Sequence ID No.: 1

```
                10 20 30 40 50
MVMIISTMEP QVSNGPTSNT SNGPSSNNRN CPSPMQTGAT TDDSKTNLIV
                60 70 80 90 100
NYLPQNMTQE EFRSLFGSIG EIESCKLVRD KITGQSLGYG FVNYIDPKDA
              110 120 130 140 150
EKAINTLNGL RLQTKTIKVS YARPSSASIR DANLYVSGLP KTMTQKELEQ
              160 170 180 190 200
LFSQYGRIIT SRILVDQVTG VSRGVGFIRF DKRIEAEEAI KGLNGQKPSG
              210 220 230 240 250
ATEPITVKFA NNPSQKSSQA LLSQLYQSPN RRYPGPLHHQ AQRFRLDNLL
              260 270 280 290 300
NMAYGVKRLM SGPVPPSACP PRFSPITIDG MTSLVGMNIP GHTGTGWCIF
              310 320 330 340 350
VYNLSPDSDE SVLWQLFGPF GAVNNVKVIR DFNTNKCKGF GFVTMTNYDE
              360 370 380
AAMAIASLNG YRLGDRVLQV SFKTNKAHKS
```

### REFERENCES

1. Adamus, G. (2009) Autoantibody targets and their cancer relationship in the pathogenicity of paraneoplastic retinopathy. Autoimmun Rev, 8, 410-414.
2. Adamus, G. et al. (1996) The occurrence of serum autoantibodies against enolase in cancer-associated retinopathy. Clin Immunol Immunopathol, 78, 120-129.
3. Carpentier, A. F. et al. (1998) Absence of HuD gene mutations in paraneoplastic small cell lung cancer tissue. Neurology, 50, 1919.
4. D'Alessandro, V. et al. (2010) Molecular analysis of the HuD gene in neuroendocrine lung cancers. Lung Cancer, 67, 69-75.
5. Dalmau, J. et al. (1992) Anti-Hu—associated paraneoplastic encephalomyelitis/sensory neuronopathy. A clinical study of 71 patients. Medicine (Baltimore), 71, 59-72.
6. Dalmau, J. et al. (1990) Detection of the anti-Hu antibody in the serum of patients with small cell lung cancer—a quantitative western blot analysis. Ann. Neurol. Ann Neurol, 27, 544-552.
7. Darnell, R. B. & DeAngelis, L. M. (1993) Regression of small-cell lung carcinoma in patients with paraneoplastic neuronal antibodies. Lancet, 341, 21-22.
8. Desrosiers, R. R. & Fanelus, I. (2011) Damaged proteins bearing L-isoaspartyl residues and aging: a dynamic equilibrium between generation of isomerized forms and repair by PIMT. Curr Aging Sci, 4, 8-18.
9. Diliberto, E. J. J. & Axelrod, J. (1976) Regional and subcellular distribution of protein carboxymethylase in brain and other tissues. Journal of Neurochemistry, 26, 1159-1165.
10. Doss, S. et al. (2014) High prevalence of NMDA receptor IgA/IgM antibodies in different dementia types. Ann Clin Transl Neurol, 1, 822-832.
11. Doyle, H. A. et al. (2003) A failure to repair self-proteins leads to T cell hyperproliferation and autoantibody production. Journal of Immunology, 171, 2840-2847.
12. Doyle, H. A. et al. (2006) Isoaspartyl post-translational modification triggers anti-tumor T and B lymphocyte immunity. Journal of Biological Chemistry, 281, 32676-32683.
13. Graus, F. et al. (1997) Anti-Hu antibodies in patients with small-cell lung cancer: association with complete response to therapy and improved survival. Journal of Clinical Oncology, 15, 2866-2872.
14. Graus, Y. F. et al. (1998) Selection of recombinant anti-HuD Fab fragments from a phage display antibody library of a lung cancer patient with paraneoplastic encephalomyelitis. Journal of Neuroimmunology, 82, 200-209.
15. Griffith, S. C. et al. (2001) Crystal structure of a protein repair methyltransferase from Pyrococcus furiosus with its L-isoaspartyl peptide substrate. Journal of Molecular Biology, 313, 1103-1116.
16. Honnorat, J. et al. (2009) Onco-neural antibodies and tumour type determine survival and neurological symptoms in paraneoplastic neurological syndromes with Hu or CV2/CRMP5 antibodies. J Neurol Neurosurg Psychiatry, 80, 412-416.
17. Howlader, N. et al. (2013) SEER Cancer Statistics Review, 1975-2011. National Cancer Institute. Bethesda, Md., http://seer.cancer.gov/csr/1975_2011/, based on November 2013 SEER data submission, posted to the SEER web site, April 2014.
18. Johnson, B. A. et al. (1989) Formation of isoaspartate at two distinct sites during in vitro aging of human growth hormone. Journal of Biological Chemistry, 264, 14262-14271.
19. Kalemkerian, G. P. (2014) Chemotherapy for small-cell lung cancer. Lancet Oncol, 15, 13-14.
20. Kazarian, M. et al. (2009) Immune response in lung cancer mouse model mimics human anti-Hu reactivity. Journal of Neuroimmunology, 217, 38-45.
21. Kazarian, M. & Laird-Offringa, I. A. (2011) Small-cell lung cancer-associated autoantibodies: potential applications to cancer diagnosis, early detection, and therapy. Molecular Cancer, 10, 33.
22. Kern, R. et al. (2005) Protein isoaspartate methyltransferase is a multicopy suppressor of protein aggregation in Escherichia coli. Journal of Bacteriology, 187, 1377-1383.
23. Kim, E. et al. (1997) Deficiency of a protein-repair enzyme results in the accumulation of altered proteins, retardation of growth, and fatal seizures in mice. Proceedings of the National Academy of Sciences of the United States of America, 94, 6132-6137.
24. Kobayashi, M. et al. (2007) Long-term survival of a patient with small cell lung cancer associated with cancer-associated retinopathy. Lung Cancer, 57, 399-403.
25. Mamula, M. J. (1998) Epitope spreading: the role of self peptides and autoantigen processing by B lymphocytes. Immunological Reviews, 164, 231-239.
26. Mamula, M. J. et al. (1999) Isoaspartyl post-translational modification triggers autoimmune responses to self-proteins. Journal of Biological Chemistry, 274, 22321-22327.
27. Manley, G. T. et al. (1995) Hu antigens: reactivity with Hu antibodies, tumor expression, and major immunogenic sites. Annals of Neurology, 38, 102-110.
28. Mizobuchi, M. et al. (1994) Tissue-specific expression of isoaspartyl protein carboxyl methyltransferase gene in rat brain and testis. Journal of Neurochemistry, 62, 322-328.
29. Monstad, S. E. et al. (2004) Hu and voltage-gated calcium channel (VGCC) antibodies related to the prognosis of small-cell lung cancer. Journal of Clinical Oncology, 22, 795-800.
30. Ohwada, A. et al. (1999) DNA vaccination against HuD antigen elicits antitumor activity in a small-cell lung cancer murine model. American Journal of Respiratory Cell and Molecular Biology, 21, 37-43.
31. Paranandi, M. V. & Aswad, D. W. (1995) Spontaneous alterations in the covalent structure of synapsin I during in vitro aging. Biochemical and Biophysical Research Communications, 212, 442-448.
32. Pulido, M. A., DerHartunian, M. K., Sehgal, P. & Laird-Offringa, I. A. (2016) Neuronal ELAVL proteins are prone to isoaspartylation. Manuscript submitted to Data in Brief.
33. Qin, Z. et al. (2014) Isoaspartyl protein damage and repair in mouse retina. Invest Ophthalmol Vis Sci, 55, 1572-1579.
34. Sekido, Y. et al. (1994) Molecular analysis of the HuD gene encoding a paraneoplastic encephalomyelitis antigen in human lung cancer cell lines. Cancer Research, 54, 4988-4992.
35. Shimizu, T. et al. (2000) Isoaspartate formation and neurodegeneration in Alzheimer's disease. Archives of Biochemistry and Biophysics, 381, 225-234.
36. Sillevis Smit, P. et al. (1996) The HuD paraneoplastic protein shares immunogenic regions between PEM/PSN patients and several strains and species of experimental animals. Journal of Neuroimmunology, 71, 199-206.
37. Sillevis Smitt, P. A. et al. (1995) Immunization with the paraneoplastic encephalomyelitis antigen HuD does not cause neurologic disease in mice. Neurology, 45, 1873-1878.
38. Sodeyama, N. et al. (1999) Pattern of epitopic reactivity of the anti-Hu antibody on HuD with and without paraneoplastic syndrome. J Neurol Neurosurg Psychiatry, 66, 97-99.
39. Totland, C. et al. (2011) CDR2 antigen and Yo antibodies. Cancer Immunol Immunother, 60, 283-289.
40. Winter, S. F. et al. (1993) Antibodies against autologous tumor cell proteins in patients with small-cell lung cancer: association with improved survival. J. Natl. Cancer Inst. J Natl Cancer Inst, 85, 2012-2018.
41. Yamamoto, A. et al. (1998) Deficiency in protein L-isoaspartyl methyltransferase results in a fatal progressive epilepsy. Journal of Neuroscience, 18, 2063-2074.
42. Yang, M. L. et al. (2006) Intracellular protein modification associated with altered T cell functions in autoimmunity. Journal of Immunology, 177, 4541-4549.
43. Zhu, J. X. et al. (2006) Protein repair in the brain, proteomic analysis of endogenous substrates for protein L-isoaspartyl methyltransferase in mouse brain. Journal of Biological Chemistry, 281, 33802-33813.
44. Zirah, S. et al. (2006) Structural changes of region 1-16 of the Alzheimer disease amyloid beta-peptide upon zinc binding and in vitro aging. Journal of Biological Chemistry, 281, 2151-2161.

1. A method of treating a cancer or inducing an immune response in a subject suffering from a cancer, comprising administering to the subject an effective amount of an isoaspartylated protein or a fragment thereof, or an antibody or antigen binding fragment that binds specifically to an isoaspartylated protein or a fragment thereof.

2. The method of claim 1, wherein the cancer is a solid tumor.

3. The method of claim 1, wherein the cancer is a cancer in tissues that would not normally express neuronal proteins.

4. The method of claim 1, wherein the cancer is selected from small cell lung cancer (SCLC), breast cancer, melanoma, and a gynecological cancer.

5. The method of claim 1, wherein the protein is an embryonic lethal altered visual system-like RNA-binding protein (Hu protein, optionally HuD protein).

6. The method of claim 1, wherein the protein comprises an isoaspartylated residue.

7. The method of claim 1, wherein the fragment comprises an amino acid capable of isoAsp conversion.

8. The method of claim 1, wherein the fragment comprises an isoaspartylated residue.

9. The method of claim 1, wherein the fragment comprises an RNA recognition motif 1 (RRM1).

10. The method of claim 1, wherein the antibody is a monoclonal antibody or an antigen binding fragment of each thereof.

11. The method of claim 10, wherein the monoclonal antibody is humanized or a polyclonal antibody.

12. (canceled)

13. The method of claim 1, wherein the treatment comprises a therapy selected from the group of a first-line therapy, a second-line therapy, a third-line therapy, a fourth-line therapy or a fifth-line therapy.

14. The method of claim 1, further comprising administering a further effective amount of the isoaspartylated protein or the fragment thereof, or the antibody that binds specifically to the isoaspartylated protein or the fragment thereof.

15. A composition, comprising:
a. an isoaspartylated protein or a fragment thereof, or an equivalent of each thereof, or
b. an antibody that binds specifically to an isoaspartylated protein or a fragment thereof;
and optionally a carrier.

16. The composition of claim 15, wherein the protein is a Hu protein or an HuD protein.

17. The composition of claim 15, wherein the protein comprises an isoaspartylated residue.

18. The composition of claim 15, wherein the fragment comprises an amino acid capable of isoAsp conversion.

19. The composition of claim 15, wherein the fragment comprises an isoaspartylated residue.

20. The composition of claim 15, wherein the fragment comprises an RRM1.

21.-23. (canceled)

24. A method to monitor the therapy of claim 1, comprising contacting a sample isolated from the subject with an effective amount of an antibody or antigen binding fragment thereof, and assaying for binding of the antibody or fragment to a polypeptide or protein in the sample, wherein the polypeptide or protein specifically to an isoaspartylated protein or a fragment thereof.

25.-28. (canceled)
